(19) United States
(12) Patent Application Publication
(10) Pub. No.: US 2026/0120886 A1
(43) Pub. Date: Apr. 30, 2026

(54) SYSTEMS AND METHODS FOR PERFORMING SERIAL DISEASE TESTING

(71) Applicant: GRAIL, Inc., Menlo Park, CA (US)

(72) Inventors: Oliver Claude VENN, San Francisco, CA (US); Joseph MARCUS, Venice, CA (US); John F. BEAUSANG, Menlo Park, CA (US); Samuel MESIROV, Sunnyvale, CA (US); Alexander P. FIELDS, Burlingame, CA (US)

(73) Assignee: GRAIL, Inc., Menlo Park, CA (US)

(21) Appl. No.: 19/375,545

(22) Filed: Oct. 31, 2025

Related U.S. Application Data
(60) Provisional application No. 63/714,523, filed on Oct. 31, 2024.

(51) Int. Cl. G16H 50/30

(52) U.S. Cl. CPC G16H 50/30

(57) ABSTRACT

Systems and methods of the disclosure may include a computer-implemented method, the computer-implemented method including: receiving, at a computer system, nucleic acid sequencing data derived from a methylation assay performed on a biological sample associated with at least one subject; computing, using a processor associated with the computer system, a beta value matrix based on the nucleic acid sequencing data, wherein the beta value matrix comprises one or more missing beta values; addressing, using the processor, the one or more missing beta values in the beta value matrix using a missing beta value completion approach; identifying, using the processor, one or more principal components in the completed beta value matrix; and training, using the one or more principal components in combination with a predetermined set of clinical variables, a classifier to predict a survival outcome for a target subject associated with a disease type.

### CROSS-REFERENCE TO RELATED APPLICATION

This application claims the benefit of priority of U.S. Provisional Patent Application No. 63/714,523, filed Oct. 31, 2024, the entirety of which is incorporated herein by reference.

### TECHNICAL FIELD

The present disclosure relates generally to the field of disease detection and diagnostics, more specifically, to systems and methods for improving the sensitivity and positive predictive value (PPV) of multi-cancer early detection (MCED) tests.

### BACKGROUND

Conventional single time point MCED tests, while useful in identifying potential cancer signals in a population, suffer from a limited ability to distinguish between false positives and true positives effectively. This limitation may lead to unnecessary follow-ups, emotional distress, and healthcare costs associated with the workup of individuals who do not have cancer. Observational data indicates that a portion of initial test-positive individuals subsequently test negative upon repeat testing after time has passed.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

Observational data indicates that a portion of initial test-positive individuals subsequently test negative upon repeat testing after a relatively short period of time has passed, suggesting an opportunity to improve the specificity and sensitivity of MCED testing. According to certain aspects of the disclosure, systems and methods are described for utilizing a sequential testing protocol to better distinguish false positives from true positives in MCED tests. The sequential testing protocol may be implemented to improve the positive predictive value of certain MCED tests without substantially modifying the physical or chemical properties of the test and/or by optimizing one or more machine-learned models used in the test.

In one aspect, a computer-implemented method for improving the accuracy of cancer screening in a population is disclosed. The computer-implemented method may include: receiving, at a computer system, a first cancer test result associated with a first test performed on a first sample associated with a subject at a first time, wherein the first cancer test result is one of: a cancer signal detected (CSD) result, an indeterminate signal result, or a non-cancer signal detected (NCSD) result; generating, responsive to receiving the indeterminate signal result, an instruction to perform a second test on the subject at a second time after a predetermined time interval; receiving, at the computer system, a second cancer test result associated with the second test performed on a second sample associated with the subject at the second time, wherein the second cancer test result is one of: a persistent CSD result or the NCSD result; and identifying, using a processor associated with the computer system, the subject as having cancer if the second cancer test result is the persistent CSD result; or identifying, using the processor, the subject as not having cancer if the second cancer test result is the NCSD result.

In another aspect, a system is disclosed. The system may include: one or more processors; one or more computer readable media storing instructions that are executable by the one or more processors to perform operations including: receiving a first cancer test result associated with a first test performed on a first sample associated with a subject at a first time, wherein the first cancer test result is one of: a cancer signal detected (CSD) result, an indeterminate signal result, or a non-cancer signal detected (NCSD) result; generating, responsive to receiving the indeterminate signal result, an instruction to perform a second test on the subject at a second time after a predetermined time interval; receiving a second cancer test result associated with the second test performed on a second sample associated with the subject at the second time, wherein the second cancer test result is one of: a persistent CSD result or the NCSD result; and identifying the subject as having cancer if the second cancer test result is the persistent CSD result; or identifying the subject as not having cancer if the second cancer test result is the NCSD result.

In yet another aspect, a non-transitory computer-readable medium is disclosed. The non-transitory computer-readable medium may store computer-executable instructions which, when executed by a system, cause the system to perform operations including: receiving, at the system, a first cancer test result associated with a first test performed on a first sample associated with a subject at a first time, wherein the first cancer test result is one of: a cancer signal detected (CSD) result, an indeterminate signal result, or a non-cancer signal detected (NCSD) result; generating, responsive to receiving the indeterminate signal result, an instruction to perform a second test on the subject at a second time after a predetermined time interval; receiving, at the system, a second cancer test result associated with the second test performed on a second sample associated with the subject at the second time, wherein the second cancer test result is one of: a persistent CSD result or the NCSD result; and identifying, using a processor associated with the system, the subject as having cancer if the second cancer test result is the persistent CSD result; or identifying, using the processor, the subject as not having cancer if the second cancer test result is the NCSD result.

In yet another aspect, a computer-implemented method for enhancing a positive predictive value of cancer screening in a population is disclosed. The computer-implemented may include: receiving, at a computer system, a first cancer test result associated with a subject for a first cancer test, wherein the first cancer test was performed at a first time; categorizing, using a processor associated with the computer system and based on the first cancer test result, the subject into at least one of a first group, a second group, and a third group, wherein the first group corresponds to a cancer signal value above a first threshold value, wherein the second group corresponds to a cancer signal value below a second threshold value, and wherein the third group corresponds to a cancer signal value between the first threshold value and the second threshold value; generating, using the processor, a first recommendation to perform a diagnostic workup if the first cancer test result is associated with the first group; generating, using the processor, a second recommendation to perform a second test on the subject if the first cancer test result is associated with the third group; receiving, at the computer system, a second cancer test result associated with the subject for a second cancer test, wherein the second cancer test result is one of: a persistent cancer signal detected (CSD) result or a non-cancer signal detected (NCSD) result; and identifying, using the processor, the subject as having cancer if the second cancer test result is the persistent CSD result; or identifying, using the processor, the subject as not having cancer if the second cancer test result is the NCSD result.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate several embodiments and together with the description, serve to explain the principles of the disclosure.

FIG. 1 depicts a graph that illustrates the longitudinal data from a sequential MCED testing program, according to one or more aspects of the present disclosure.

FIG. 2 depicts a graph showing a receiver operating characteristic (ROC) curve that demonstrates the MCED test's performance at various decision thresholds, according to one or more aspects of the present disclosure.

FIG. 3A depicts a graph that shows the overall distribution of cfDNA tumor fraction (TF) values across study cohorts, according to one or more aspects of the present disclosure.

FIG. 3B depicts a graph that further separates the TF distributions associated with FIG. 18A by cancer status, according to one or more aspects of the present disclosure.

FIG. 4 depicts graphs that collectively illustrate how TF influences cancer detection performance across different cancer types and clinical stages, according to one or more aspects of the present disclosure.

FIG. 5 depicts a graph containing scatter and violin plots that illustrate how undetected cancer samples are distributed relative to percentile-ranked non-cancer scores, according to one or more aspects of the present disclosure.

FIG. 6 depicts a graph that illustrates how the fraction of detected cancers change over time before diagnosis for two longitudinal studies, according to one or more aspects of the present disclosure.

FIG. 7 depicts a graph that illustrates the relationship between TF and the probability of a cancer-like signal, according to one or more aspects of the present disclosure.

FIG. 8 depicts a graph that illustrates the general relationship between cancer progression and the amount of ctDNA shed into the bloodstream, according to one or more aspects of the present disclosure.

FIG. 9 depicts a graph that illustrates how repeated MCED testing over time may capture the dynamics of tumor DNA shedding and improve early detection at the individual level, according to one or more aspects of the present disclosure.

FIG. 10 depicts a graph that illustrates how improvements in MCED test performance may enable earlier identification of cancer and thereby improve clinical outcomes, according to one or more aspects of the present disclosure.

FIG. 11 demonstrates diagrams that illustrate how longitudinal blood-based testing reveals increasing signal detection as cancer diagnosis approaches, according to one or more aspects of the present disclosure.

FIG. 12 depicts a graph that illustrates how TMeF increases over time as cancer progresses toward clinical diagnosis, according to one or more aspects of the present disclosure.

FIG. 13 depicts a diagram that illustrates a conceptual framework for improving MCED test performance through a two-stage testing strategy, according to one or more aspects of the present disclosure.

FIG. 14 depicts an exemplary workflow diagram for improving the accuracy of cancer screening in a population, according to one or more aspects of the present disclosure.

FIG. 15A depicts an exemplary computer system for executing the methods described herein, according to one or more aspects of the present disclosure.

FIG. 15B depicts an exemplary software platform for executing the methods described herein.

FIG. 16 depicts a diagram summarizing clinical outcomes for individuals who initially received a cancer signal detected result from an MCED test and subsequently underwent retesting, according to one or more aspects of the present disclosure.

FIG. 17 depicts a diagram showing a flowchart for a sequential testing algorithm configured to categorize individuals based on their initial cancer signal and inform a subsequent retesting strategy, according to one or more aspects of the present disclosure.

FIG. 18 depicts a graph illustrating how the overall false positive rate (FPR) behaves in a sequential testing framework for multi-cancer detecting, according to one or more aspects of the present disclosure.

FIG. 19 depicts graphs that collectively illustrate how the FPR behaves in a sequential testing framework across multiple settings for the high-threshold test's false positive rate, according to one or more aspects of the present disclosure.

FIG. 20 depicts a graph that illustrates how overall sensitivity behaves in a sequential testing framework for multi-cancer detection, according to one or more aspects of the present disclosure.

FIG. 21 depicts a diagram that illustrates the conceptual workflow for a power simulation used to evaluate differences in FPR between sequential tests, according to one or more aspects of the present disclosure.

FIG. 22 depicts a graph that illustrates the results of a power simulation evaluating the ability to detect a difference in FPR between two sequential tests using an analytic calculation under a normal approximation, according to one or more aspects of the present disclosure.

FIG. 23 depicts a graph that illustrates the results of a power simulation evaluating the ability to detect a difference in true positive rate (TPR), or sensitivity, between two sequential tests using an analytic calculation under a normal approximation, according to one or more aspects of the present disclosure.

FIG. 24 depicts graphs that collectively illustrate the results of a paired study design simulation assessing the FPR for sequential testing system compared to a single point-in-time MCED test, according to one or more aspects of the present disclosure.

FIG. 25 depicts a graph that illustrates a paired study design simulation evaluating the TPR performance of a sequential testing system compared to a single MCED test, according to one or more aspects of the present disclosure.

FIG. 26 depicts an example computing system, according to one or more embodiments of the present disclosure.

### DETAILED DESCRIPTION OF EMBODIMENTS

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

Cancer represents a prominent worldwide public health problem. The United States alone in 2015 had a total of 1,658,370 cases reported. Screening programs and early diagnosis have an important impact in improving disease-free survival and reducing mortality in cancer patients. For example, early screening of colorectal cancer (CRC) has led to almost a 50% decrease in CRC incidence and mortality in the U.S. This reduction is consistent with stage-dependent survival rates for the cancer, which decrease from 94% in stage 1 CRC to 11% in stage 4 CRC. However, there are two major challenges with early cancer detection: patient compliance and poor sensitivity.

Advantageously, increasing knowledge of the molecular pathogenesis of cancer and the rapid development of next generation sequencing techniques are advancing the study of early molecular alterations involved in cancer development in body fluids. Specific genetic and epigenetic alterations associated with such cancer development are found in cell-free DNA (cfDNA) in plasma, serum, and urine. Such alterations can potentially be used as diagnostic biomarkers for several types of cancers. Advantageously, non-invasive sampling methods, such as so-called ‘liquid biopsies,’ can foster patient compliance, as they are easier, quicker, and less expensive to perform.

Cell-free DNA (cfDNA) can be found in serum, plasma, urine, and other body fluids enabling the ‘liquid biopsy,’ which represents a snapshot of the genomic makeup of many different tissues in the subject, including diseased tissues. cfDNA originates from necrotic or apoptotic cells, and it is generally released by all types of cells. cfDNA contains specific tumor-related alterations, such as mutations, methylation, and copy number variations (CNVs), thus comprising circulating tumor DNA (ctDNA).

However, because cfDNA represents DNA released from a wide range of tissues, including healthy tissues and white blood cells undergoing hematopoiesis, the challenge remains to be able to differentiate the signal originating from a disease tissue, such as cancer, from signals originating from germline cells. In fact, in most cancer patients, the majority of cfDNA is from healthy cells, e.g., greater than 80%, 90%, 95%, or more. cfDNA signals can be enriched, for example, bioinformatically by identifying variant alleles having allele fractions that do not adhere to typical 1:1 ratios, as seen for heterozygous alleles in the germline. cfDNA signals can also be enriched based on the size of the cfDNA being sequenced, because it has been observed that cfDNA originating from cancerous tumor is, on average, shorter in length than cfDNA originating from germline cells.

Unfortunately, to date, the majority of cfDNA diagnostic studies are focused on advanced tumor stages. The application of cfDNA-based diagnostic assays for identification of early malignant disease stages is less well documented. Although early stage cancer detection works on the same principals as later stage cancer detection, there are several impediments that are unique to early stage detection. These include lower frequency and volume of aberrations, potentially confounding phenomena such as clonal expansions of non-tumorous tissues or the accumulation of cancer-associated mutations with age, and the incomplete insight into driver alterations.

In blood, apoptosis is a frequent event that determines the amount of cfDNA. In cancer patients, however, the amount of cfDNA can also be influenced by necrosis. Since apoptosis seems to be the main release mechanism, circulating cfDNA has a size distribution that reveals an enrichment in short fragments of about 167 bp, corresponding to nucleosomes generated by apoptotic cells.

As used herein, the term “methylation” refers to a modification of deoxyribonucleic acid (DNA) where a hydrogen atom on the pyrimidine ring of a cytosine base is converted to a methyl group, forming 5-methylcytosine. Methylation can occur at dinucleotides of cytosine and guanine referred to herein as “CpG sites”. In other instances, methylation may occur at a cytosine not part of a CpG site or at another nucleotide that's not cytosine; however, these are rarer occurrences. In this present disclosure, methylation can be discussed in reference to CpG sites for the sake of clarity. Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

Various challenges arise in the identification of anomalously methylated cfDNA fragments. First, determining a subject's cfDNA to be anomalously methylated can hold weight in comparison with a group of control subjects, such that if the control group is small in number, the determination can lose confidence with the small control group. Additionally, among a group of control subjects', methylation status can vary which can be difficult to account for when determining a subject's cfDNA to be anomalously methylated. On another note, methylation of a cytosine at a CpG site can causally influence methylation at a subsequent CpG site.

The principles described herein can be equally applicable for the detection of methylation in a non-CpG context, including non-cytosine methylation. Further, the methylation state vectors may contain elements that are generally vectors of sites where methylation has or has not occurred (even if those sites are not CpG sites specifically). With that substitution, the remainder of the processes described herein are the same, and consequently, the inventive concepts described herein are applicable to those other forms of methylation.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density, and proportion of methylated cytosines are examples of “methylation levels.”

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example, 5′-CHG-3′ and 5′-CHH-3′, where H is adenine, cytosine or thymine. Cytosine methylation can also be in the form of 5-hydroxymethylcytosine. Methylation of DNA can include methylation of non-cytosine nucleotides, such as N6-methyladenine.

Cancer screening aims to identify the presence of cancer at its earliest and most treatable stages. Multi-cancer early detection (MCED) tests are designed to identify cancer signals in a population with relatively low incidence rates, often by analyzing biomarkers in biological samples, such as blood samples. However, current MCED tests face a significant challenge-distinguishing true positives (i.e., actual cases of cancer) from false positives (i.e., non-cancer cases flagged as potential cancer) at a single time point. This limitation may have broad implications, resulting in unnecessary diagnostic follow-ups-potentially including invasive procedures, increased healthcare costs, and potential emotional distress for individuals receiving false-positive results and their community.

In conventional approaches, MCED tests may employ classifiers optimized to balance sensitivity (e.g., the ability to detect true cancer cases) and specificity (e.g., the ability to avoid false positives). Often, single-time-point MCED tests aim for high specificity to reduce the incidence of false positives. However, this can limit the sensitivity of the test, potentially missing cancers that could have been identified with a broader detection threshold. Conversely, lowering the specificity threshold to increase sensitivity may lead to higher rates of false positives. While some efforts have been made to improve these outcomes, they primarily involve using additional tests at the same time point, such as reflex testing with different biomarkers or technologies. These reflex tests, while occasionally helpful, lack the ability to incorporate the natural signal dynamics over time, which is observed in real-world data as being important for distinguishing true positives from false positives. Further, while some efforts have been made to test at multiple time points, these tend to use different tests (e.g., an initial screening test and then a more nuanced test ordered after the results of the initial test are obtained; or two different testing modalities such as an initial liquid biopsy testing and then an imaging-based test ordered after the results of the liquid biopsy test are obtained) at different time points, or they use tests at far apart time points that are intended to track whether a diagnosis has changed over time, rather than using both tests at both time points to determine an initial diagnosis. Additionally, currently available or studied testing modalities are not able to share information between the two tests at different time points. For example, current testing modalities are not able to take into consideration the results of a first test when determining the result of the second test.

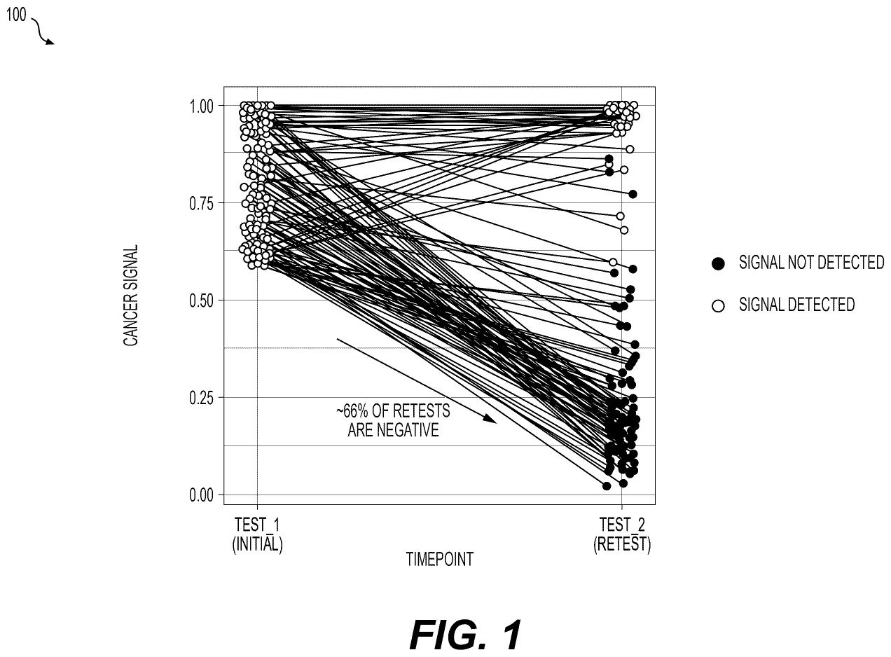

As an example of the foregoing, turning now to FIG. 1, graph 100 illustrates the longitudinal data from a sequential MCED testing program, showing cancer signal dynamics between an initial test result (Test 1) at a first time point and a retest result (Test 2) 3-6 months after the first time point. On the x-axis, Test 1 and Test 2 represent two time points, while the y-axis shows the classifier's normalized outputted cancer signal, ranging from 0 to 1. Higher values on the y-axis correspond to a stronger cancer signal, suggesting a greater likelihood of an individual having cancer. The data included in graph 100 highlights the observation that, in this cohort, among individuals with a positive cancer signal in the initial test (represented in open white circles on the left side of graph 100), approximately 66% tested negative on the subsequent test (represented by black closed circles on the right side of the graph). This suggests that many initial positives are likely false positives, as these signals diminish or disappear over time without any cancer diagnosis in the intervening period. This pattern may imply that the initial positives are enriched with naturally occurring transient signals or short-term confounding conditions that decay, marking them as false positives. However, a subset of positive cancer signals show sustained or even increasing signal levels upon retesting. These cases are likely true positives, potentially representing either individuals with cancer or those with persistent biological markers indicative of cancer.

Graph 100 categorizes test results into three primary behaviors across time. The first category may include samples that have a persistent high signal, in which the sample maintains a strong signal (e.g., remaining near the “1” on the y-axis) across both tests. These samples are highly likely to be true positives, as their relative signal stability suggests ongoing cancer-related biological activity. The second category encompasses those samples that exhibit a moderate signal with decay, which includes samples that initially show moderate cancer signal levels close to the decision threshold after the first test, but drop below this threshold after administration of the second test. These cases, moving from signal detected to undetected, are less likely to represent true cancer cases. The third category corresponds to those samples exhibiting a significant signal drop in cancer signal between the first and second tests (e.g., moving from a detected result to nearly zero). This group of individuals is especially likely to consist of false positives, as their signal quickly diminishes to levels that would not warrant a cancer diagnosis. While helpful, this data reflects test results obtained at time points approximately 3-6 months apart from each other, which may not reflect a practical amount of time lapse for providing an initial diagnosis to an individual especially given the aggressiveness of many cancers and the assumed asymptomatic nature of the patient population for a screening test.

The concepts described herein introduce a novel sequential testing approach that may use time as an additional dimension in disease, e.g., cancer, detection. Instead of relying on a single static test result, this method leverages the observation that false-positive signals often decay over time, whereas true positive cancer signals tend to persist or increase. By implementing a two-stage testing protocol, the system may implement the same MCED test at an initial time point and then again at a subsequent time point (e.g., 1-8 weeks later, e.g., 3-6 weeks, 4-6 weeks, etc.) to determine whether a cancer signal detected at the first time point has persisted to the second time point. In other aspects, a two-stage testing protocol may entail a system that performs a first, initial MCED test at a first time point with a relatively lower specificity threshold resulting in more initial false positives and a wider positive result window. The initial test thus casts a broader net for potential positives. This initial test may identify individuals with an intermediate cancer signal, who are then reflexed to a follow-on test after a short predefined interval (e.g., 1-8 weeks). In some embodiments, the only difference between the first test and the follow-on test may be the threshold for a positive or intermediate cancer signal (and resulting sensitivity). In some embodiments, the first test may be different than the second test in at least one aspect and may be optimized to identify decaying false positives while confirming persistent or increasing signals as true positives. This sequential strategy may allow the test r to operate at lower specificity initially, thereby capturing more true positives yet still maintain a high overall specificity without the long-term burden of false-positive cases. In some cases, the classifier may use multiple co-optimized sub-models that are specialized for the specific stage within the sequential testing protocol.

The concepts described herein address the limitations of single time point MCED tests by providing a more flexible and efficient screening framework. By incorporating sequential testing, this method may achieve one or more of higher sensitivity and mitigating the risk of false positives. In some aspects, the initial test may allow for use of a lower positive result threshold (and related sensitivity), which may improve the test sensitivity. The second-stage test then may reduce the rate of false positives by recognizing decaying signals, which are more likely to be non-cancer cases. This approach may improve the overall PPV without sacrificing sensitivity, critical characteristics for a test available for use for a large population of individuals. Moreover, the sequential testing method may reduce the need for unnecessary diagnostic follow-ups, thus lowering healthcare costs and providing a more precise cancer detection method that benefits patients and healthcare providers.

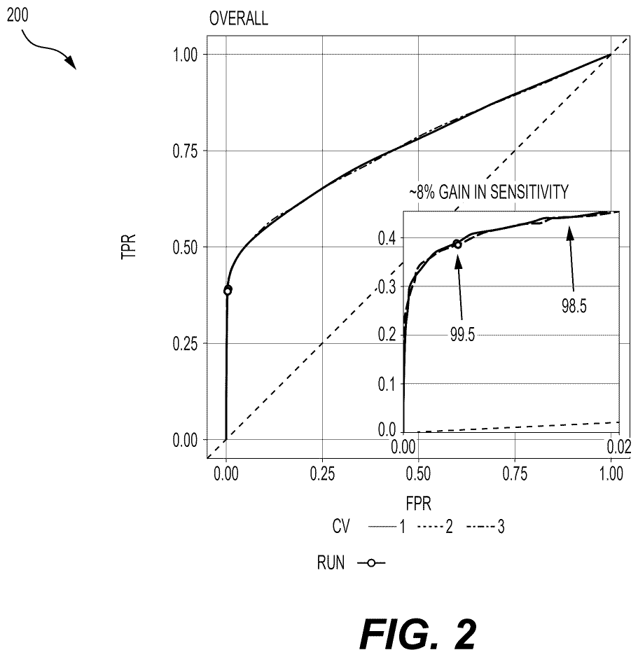

For instance, referring now to FIG. 2, graph 200 depicts a Receiver Operating Characteristic (ROC) curve, a plot that illustrates the trade-off between the true positive rate on the y-axis and the false positive rate on the x-axis across different threshold settings for a cancer classifier. As indicated above, one aim of this disclosure is to bolster sensitivity (i.e., the true positive rate) while managing an acceptable false positive rate, particularly aiming for a target false positive rate below 1%, and ideally around or below 0.5%. The ROC curve demonstrates the classifier's performance at various decision thresholds, where each point on the curve represents a sensitivity-specificity trade-off. In an aspect, the operational threshold of an original classifier may be set to achieve a high specificity (or low false-positive rate), which is seen towards the lower left of the curve. This ensures that the false positive rate is minimized, making it suitable for a screening population where unnecessary follow-ups due to false positives may lead to significant costs and patient burden.

An inset within graph 200 highlights two specific thresholds (0.5% false positive rate at 99.5 specificity and a more relaxed 98.5 specificity), showing that by lowering the specificity threshold to allow approximately 3 times the number of false positives (i.e., widening the “funnel” of the classifier), there is a substantial gain in sensitivity, e.g., approximately 8%. This increased sensitivity may allow the classifier to detect a greater number of true cancer cases across various types, potentially enhancing the overall cancer yield of the test. Accordingly, this 8% gain in sensitivity across all cancer types suggests that with a slightly lower specificity, the classifier may capture more cancer cases. This is an appealing trade-off for an initial test, as higher sensitivity increases the likelihood of detecting cancer early, even though it comes at the cost of a modest increase in false positives. However, it is still highly desirable to drive the overall false positive rate as low as feasible (e.g., near or below 0.5%) in order to minimize the costs and risk associated with false positives. Therefore, by allowing a slightly wider initial “funnel” for detecting cancer signals, the test may achieve greater initial sensitivity, while the sequential testing model addresses the false positives by using a follow-on test, maintaining an effective and adjustable balance between sensitivity and specificity.

The disclosed systems and methods produce concrete, real-world improvements in the operation and reliability of molecular diagnostic systems. By integrating computer-implemented temporal analysis into the disease detection workflow, the system may generate more accurate and actionable diagnostic outputs than conventional static testing frameworks. These improvements are not merely abstract, but are rooted in the enhanced computational processing of sequential data, enabling the system to dynamically update classifications, reduce false positives, and improve predictive confidence. The resulting reduction in false positives translates directly to a reduction in unnecessary follow-up medical procedures, improved clinical decision-making, and efficient allocation of healthcare resources which constitute tangible benefits arising from the technological innovation itself.

The subject matter of the present disclosure will now be described more fully hereinafter with reference to the accompanying drawings, which form a part hereof, and which show, by way of illustration, specific exemplary embodiments. An embodiment or implementation described herein as “exemplary” is not to be construed as preferred or advantageous, for example, over other embodiments or implementations; rather, it is intended to reflect or indicate that the embodiment(s) is/are “example” embodiment(s). Subject matter may be embodied in a variety of different forms and, therefore, covered or claimed subject matter is intended to be construed as not being limited to any exemplary embodiments set forth herein; exemplary embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware, or any combination thereof. The following detailed description is, therefore, not intended to be taken in a limiting sense.

Throughout the specification and claims, terms may have nuanced meanings suggested or implied in context beyond an explicitly stated meaning. Likewise, the phrase “in one embodiment” or “in some embodiments,” or “in one aspect” or “in some aspects” as used herein does not necessarily refer to the same embodiment or aspect, and the phrase “in another embodiment” or “in another aspect” as used herein does not necessarily refer to a different embodiment or aspect. It is intended, for example, that claimed subject matter include combinations of exemplary embodiments in whole or in part.

Diseases referred to herein may include cancer. For instance, non-limiting hematologic malignancies referred to herein may include b-cell lymphoma, CLL_SLL, DLBCL, essential thrombocythemia, follicular lymphoma, Hodgkin lymphoma, lymphoplasmacytic, MALT NMZL, mantle cell, MDS, MGUS, plasma cell myeloma, plasma cell neoplasm, and polycythemia vera. Additionally or alternatively, non-limiting cancer types that the concepts described herein may be applied to include, for example, breast cancer, lung cancer (e.g., non-small cell lung cancer (NSCLC)), prostate cancer, colorectal cancer, renal cancer, uterine cancer, pancreatic cancer, cancer of the esophagus, a lymphoma, head and neck cancer, ovarian cancer, a hepatobiliary cancer, a melanoma, cervical cancer, multiple myeloma, leukemia, thyroid cancer, bladder cancer, and gastric cancer. Additionally, it is also important to note that although the concepts described throughout this disclosure are made in reference to cancer, these designations are for exemplary purposes only and are not intended to be limiting. Specifically, the concepts described herein may be applicable to other disease types and other disease-detecting machine-learning classifiers.

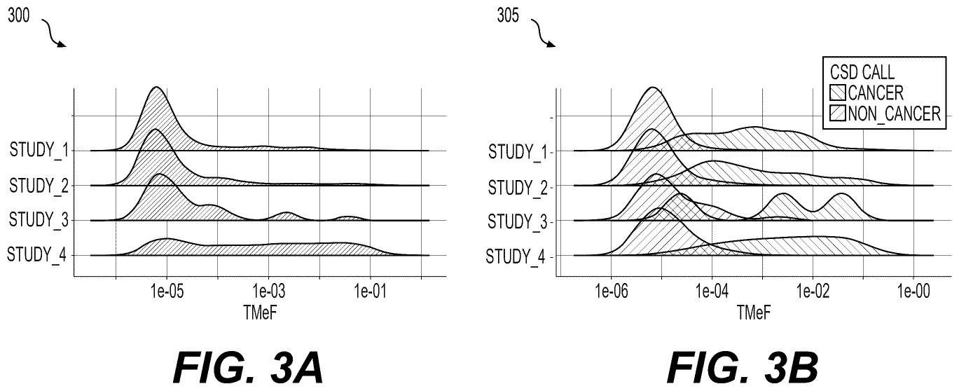

FIGS. 3A-12 explore the relationship between tumor fraction (or Tumor Methylation Fraction (TMeF)) and cancer signal as detected over time. In total, these figures and their associated discussion provide a basis for using testing across multiple time points as a mechanism to improve the performance metrics of an MCED test at population scale.

Referring now to FIG. 3A, graph 300 shows the overall distribution of cfDNA tumor fraction (TF) values across tested samples in a variety of study cohorts, illustrating that TF tends to occur in two distinct ranges rather than being evenly distributed. Most samples exhibit either very low TF values (e.g., on the order of 10⁻⁵ or below) or relatively high TF values (around 10⁻³ or greater), with few instances falling between these extremes. This bimodal pattern indicates that cfDNA samples generally fall into clear detectable or non-detectable categories, which constrains the window in which early cancer signals may be measured. In FIG. 3B, graph 305 further separates these TF distributions by cancer status, comparing samples classified as cancer (represented by falling diagonal lines) versus non-cancer (represented by rising diagonal lines). The cancer-associated samples are enriched in the higher TF range, while non-cancer samples are concentrated at the lowest TF levels.

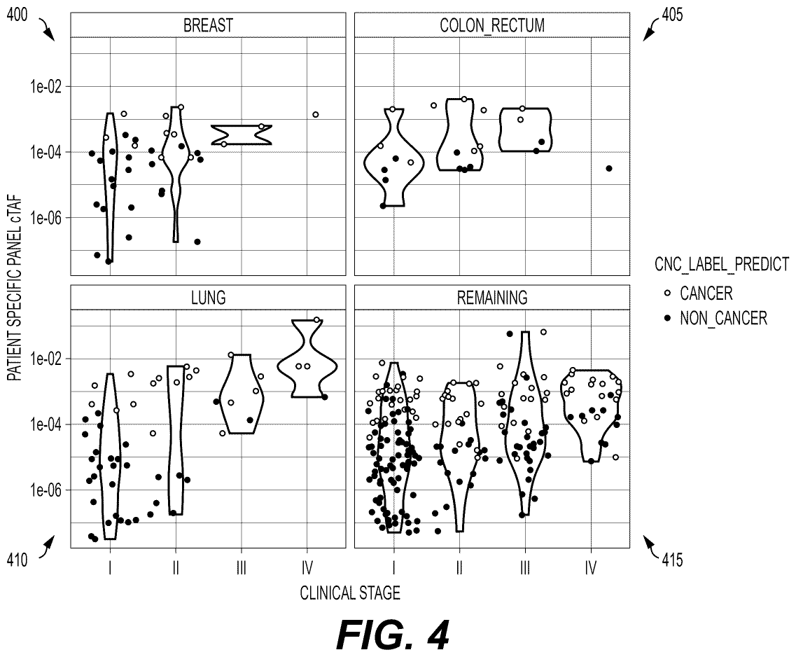

Referring now to FIG. 4, graphs 400-415 collectively illustrate how TF influences cancer detection performance across different cancer types and clinical stages, according to one or more aspects of the present disclosure. Graphs 400-415 represent breast 400, colorectal 405, lung 410, and other remaining cancer types 415, showing patient-specific TF levels on a logarithmic scale (y-axis) plotted against clinical stage (x-axis). Empty circles correspond to samples predicted as cancer, while solid circles represent non-cancer predictions. Across all cancer types, TF varies by several orders of magnitude, ranging from approximately 10⁻⁶ to 10⁻², and generally increases with clinical stage, reflecting higher tumor burden in more advanced disease. The violin plots in each graph 400-415 highlight that cancer samples tend to have higher TF values than non-cancer samples, demonstrating a strong correlation between TF and correct cancer classification. In some aspects, TF may be estimated from predetermined genomic panels with a limit of quantification around 10⁻⁵, and that cancer detection remains consistent relative to TF, underscoring its role as a key determinant of assay sensitivity and classification accuracy.

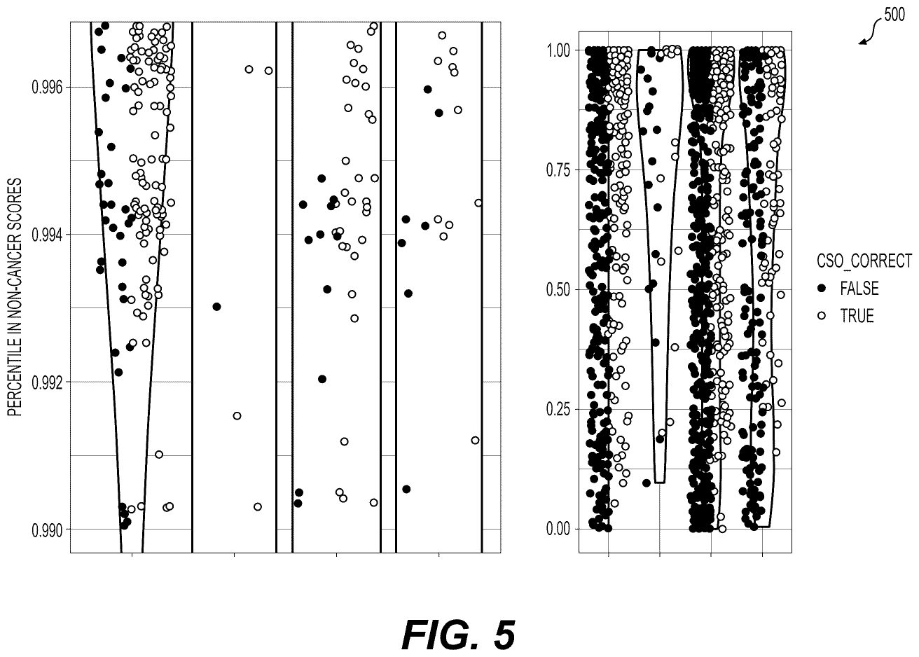

Referring now to FIG. 5, the scatter and violin plots in graph 500 illustrate how undetected cancer samples are distributed relative to percentile-ranked non-cancer scores, according to one or more aspects of the present disclosure. Examination of graph 500 reveals that most undetected cancers cluster within the uppermost percentiles of non-cancer scores, typically within a range 3× to 10× below the established LoD, indicating that they still exhibit weak but measurable cancer-associated signal. The data suggest that these cancers may be within “striking distance” of detection if either the assay's biological noise floor were lowered or the non-cancer background modeling were improved. However, research has shown that simply introducing additional data from to single point-in-time samples is unlikely to improve algorithmic detection approaches such that the biological noise floor is overcome. Furthermore, research has shown that increasing the sequencing depth of samples has diminishing returns. As described herein, one approach to re-evaluating the sample is to employ a sequential testing regime in which these borderline cases are re-evaluated within a short timeframe.

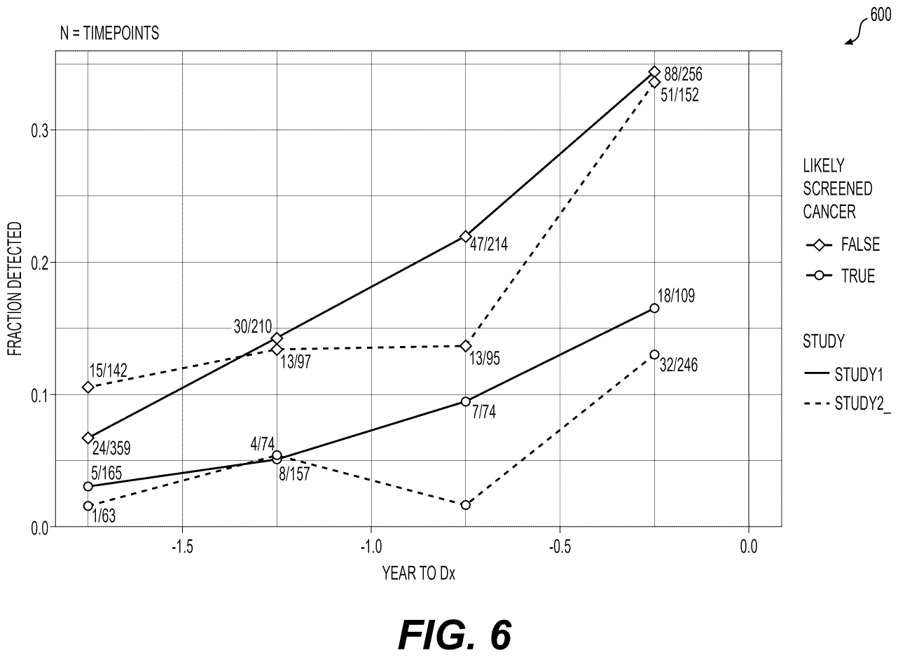

Referring now to FIG. 6, graph 600 illustrates how the fraction of detected cancers changes over time before diagnosis (Year to Dx) for two longitudinal studies, labeled STUDY1 (in solid lines) and STUDY2 (in broken lines), according to one or more aspects of the present disclosure. The x-axis represents timepoints leading up to diagnosis, while the y-axis shows the proportion of cancers detected by the assay at each interval. Results are stratified by whether cancers were likely screen-detected (represented by open circles) or not (represented by open diamonds). Across both studies, detection rates increase as the time to diagnosis decreases, indicating that as clinical diagnosis approaches, tumor burden or circulating tumor signal increases, making detection more likely. Non-screen-detected cancers (open diamonds) consistently show higher detection fractions compared to screen-detected cancers (open circles), likely reflecting larger tumor fractions or more advanced disease at detection. Overall, the figure demonstrates that assay sensitivity improves with proximity to diagnosis and varies depending on the clinical detection context.

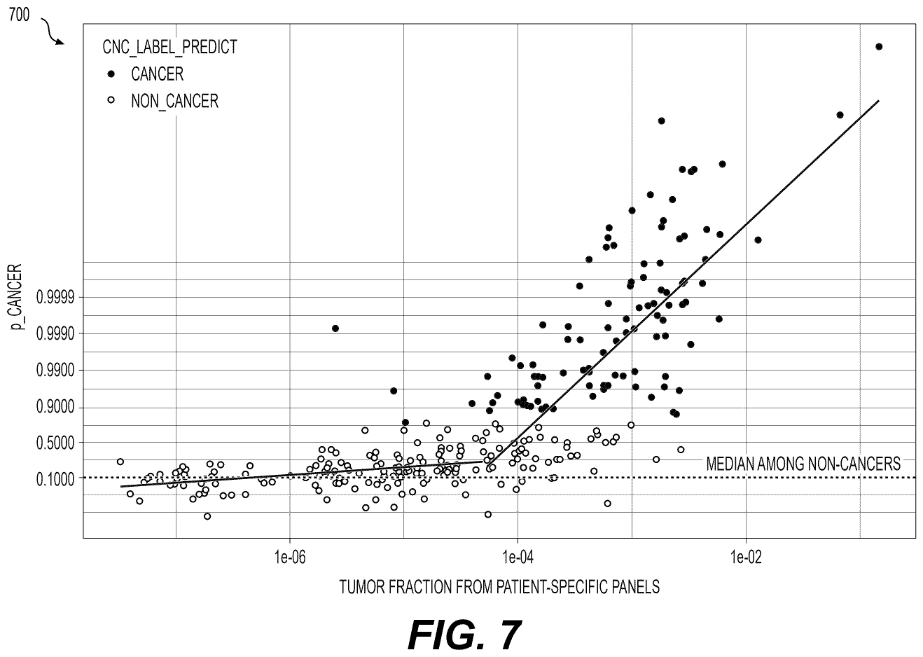

Referring now to FIG. 7, graph 700 illustrates the relationship between TF (x-axis) and the probability of a cancer-like signal (y-axis), with solid circles representing cancer samples and open circles representing non-cancer samples. As TF increases, the likelihood of a cancer-like signal also increases, as indicated by the upward trend line. However, even among non-cancer samples, a small proportion (˜1 in 10,000 DNA molecules) exhibits methylation patterns resembling those of cancer, resulting in a measurable baseline signal, e.g., the “noise floor,” represented by the dashed line marking the median among non-cancers. Graph 700 highlights that residual background noise in non-cancer cfDNA fundamentally constrains achievable detection performance at very low tumor fractions. Therefore, identifying and excluding this background noise, including on an individualized basis, is desirable. According to embodiments discussed herein, one mechanism to accomplish this is through sequential testing of cases that are near this floor with indeterminate cancer signal. As described herein, a classier can be trained or fine-tuned to accommodate this noise floor when evaluating a retested sample.

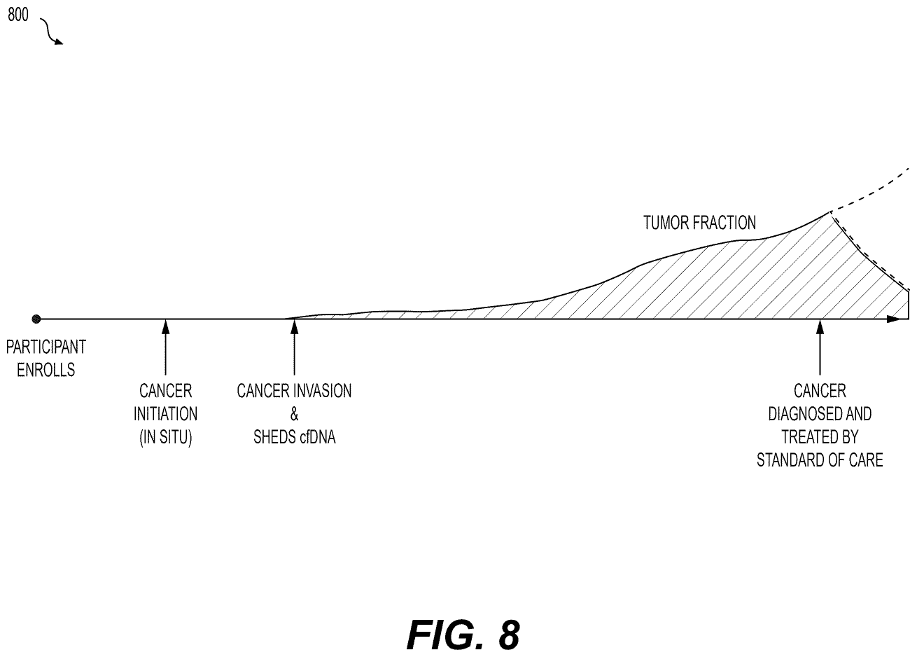

Referring now to FIG. 8, graph 800 illustrates an example of the general relationship between cancer progression and the amount of ctDNA shed into the bloodstream, represented as tumor fraction over time, according to one or more aspects of the present disclosure. The x-axis depicts the disease timeline, beginning with a participant's enrollment in a screening regime, followed by cancer initiation (in situ), invasion with cfDNA shedding, and eventual diagnosis and treatment by standard clinical methods. The region containing rising diagonal lines shows that tumor fraction remains very low or undetectable during early, localized stages of disease, but increases steadily as the tumor grows and becomes invasive. This rise reflects greater release of tumor-derived DNA into circulation as cancer cells proliferate and die. The dotted line at the far right suggests potential variability in ctDNA levels near diagnosis, influenced by biological factors or treatment.

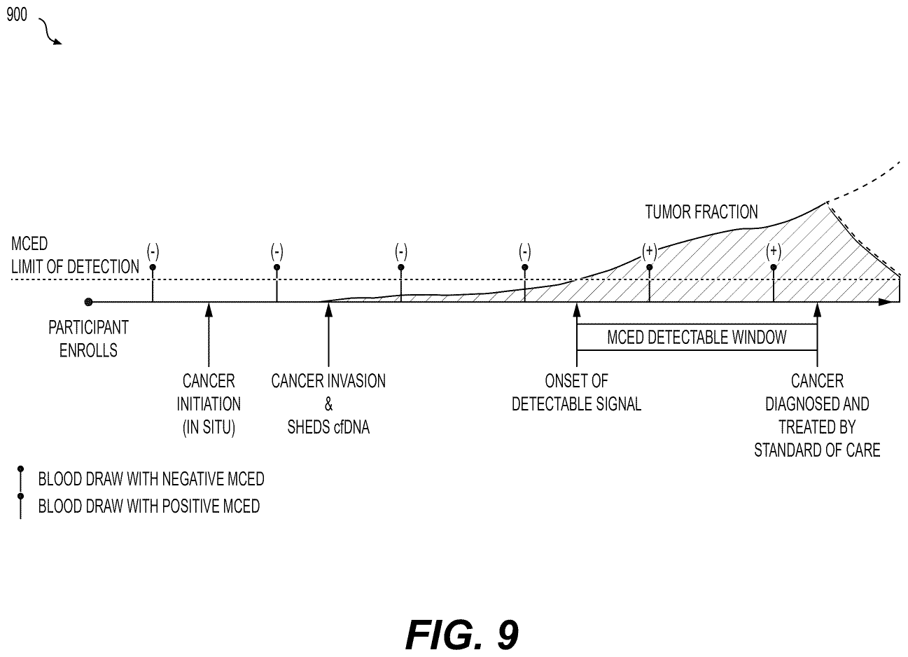

Referring now to FIG. 9, graph 900 illustrates how repeated MCED testing over time may capture the dynamics of tumor DNA shedding and improve early detection at the individual level, according to one or more aspects of the present disclosure. The x-axis represents disease progression, from participant enrollment in a screening regime, through cancer initiation and invasion, to eventual clinical diagnosis and treatment, while the region containing rising diagonal lines shows tumor fraction (the amount of tumor-derived cfDNA in blood) increasing as the cancer progresses. The dashed horizontal line marks a hypothetical MCED assay's limit of detection. Early in disease, blood draws (marked by rising diagonal lines) yield negative results because the tumor fraction remains below this detection threshold. As the tumor grows and cfDNA shedding increases, the tumor fraction surpasses the detection limit, leading to the first positive test result, marking the onset of the MCED detectable window. Subsequent blood draws within this window continue to test positive until standard diagnostic methods identify and confirm the cancer.

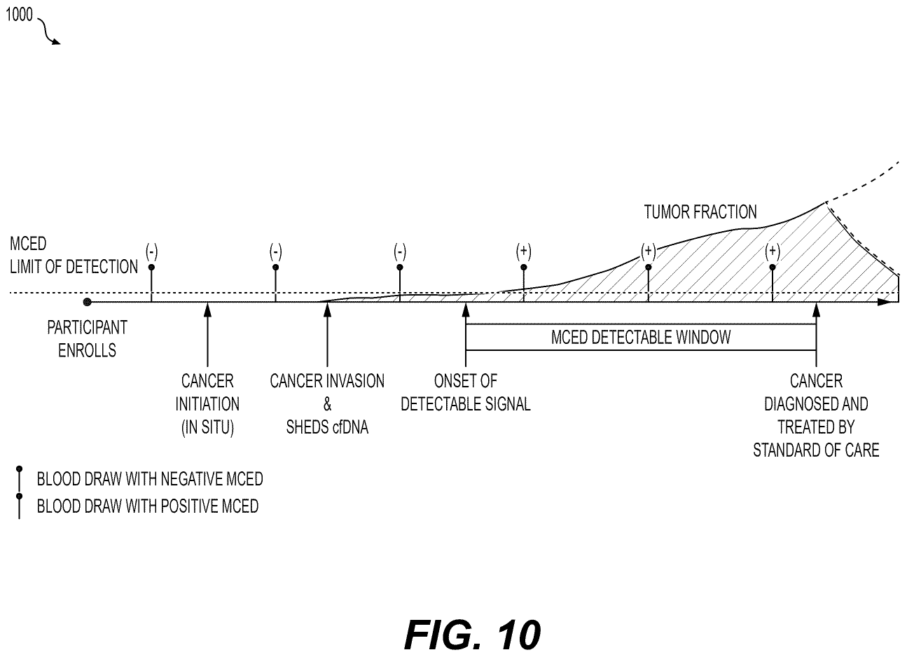

Referring now to FIG. 10, graph 1000 illustrates how improvements in MCED test performance may enable earlier identification of cancer and thereby improve clinical outcomes, according to one or more aspects of the present disclosure. The x-axis represents the disease timeline, from participant enrollment in a screening regime through cancer initiation, invasion, and eventual diagnosis and treatment, while the rising diagonal lines shaded region represents tumor fraction, or the amount of tumor-derived cfDNA circulating in blood. The dashed horizontal line marks the hypothetical MCED test's limit of detection. As the cancer progresses, tumor fraction gradually rises until it surpasses the detection threshold. In the situation represented by graph 2600, an enhanced MCED test (with a lower LOD) detects the cancer earlier in its course, indicated by the first positive blood draw (marked “(+)”) occurring sooner and at a lower tumor fraction compared to FIG. 9. Subsequent tests remain positive as the tumor continues to shed cfDNA. This shift expands the MCED detectable window, allowing cancers to be identified earlier than through standard diagnostic methods, which may lead to earlier intervention and better patient outcomes.

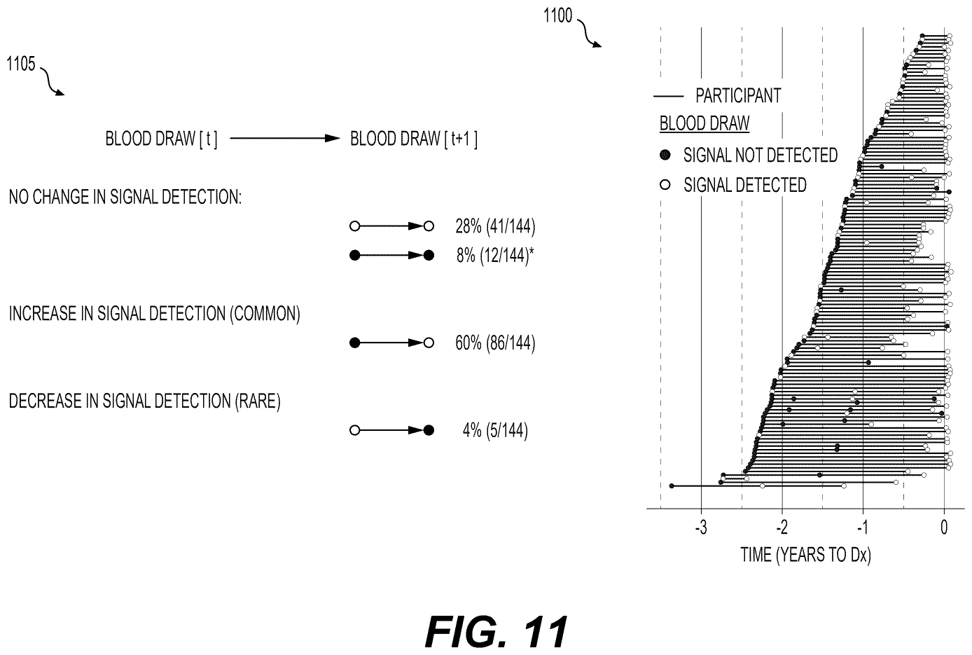

FIG. 11 demonstrates how longitudinal blood-based testing reveals increasing signal detection as cancer diagnosis approaches, according to one or more aspects of the present disclosure. Diagram 1100 shows individual participant trajectories, in which each dot represents a blood draw, e.g., a solid circle for “signal not detected” and an empty circle for “signal detected,” plotted relative to time before diagnosis (Years to Dx). Over time, the proportion of empty circles rises sharply, indicating that cancer-related cfDNA signals become more detectable as disease progresses. Panel 1105 summarizes these trends quantitatively: between consecutive blood draws, in one evaluation, 60% of participants show an increase in signal detection (transitioning from undetected to detected), while 28% show no change in detection status when signal was already present, and only 4% exhibit a decrease (signal loss). A smaller portion (8%) show no change in undetected status. These results emphasize that signal detection typically strengthens with time, supporting the idea that repeated testing can capture dynamic increases in tumor-derived cfDNA as cancers evolve toward clinical detectability.

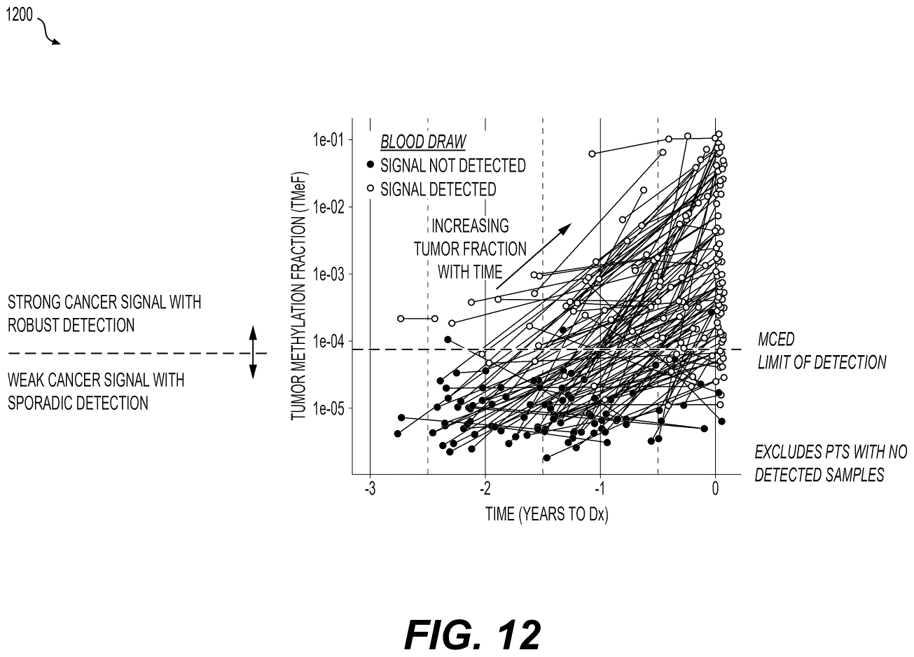

Referring now to FIG. 12, graph 1200 illustrates how TMeF increases over time as cancer progresses toward clinical diagnosis, according to one or more aspects of the present disclosure. The x-axis represents time before diagnosis (Years to Dx), while the y-axis shows TMeF on a logarithmic scale. Each dot corresponds to an individual blood draw, with a solid circle indicating “signal not detected” and an open circle indicating “signal detected.” The dashed horizontal line marks the MCED test's limit of detection. Early in the timeline, most samples fall below this threshold, showing weak or sporadic detection. As time advances, and cancer develops and/or progresses, TMeF levels rise, resulting in more frequent and consistent signal detection. The connecting lines show individual participant trajectories, demonstrating that tumor fraction tends to grow rapidly over time.

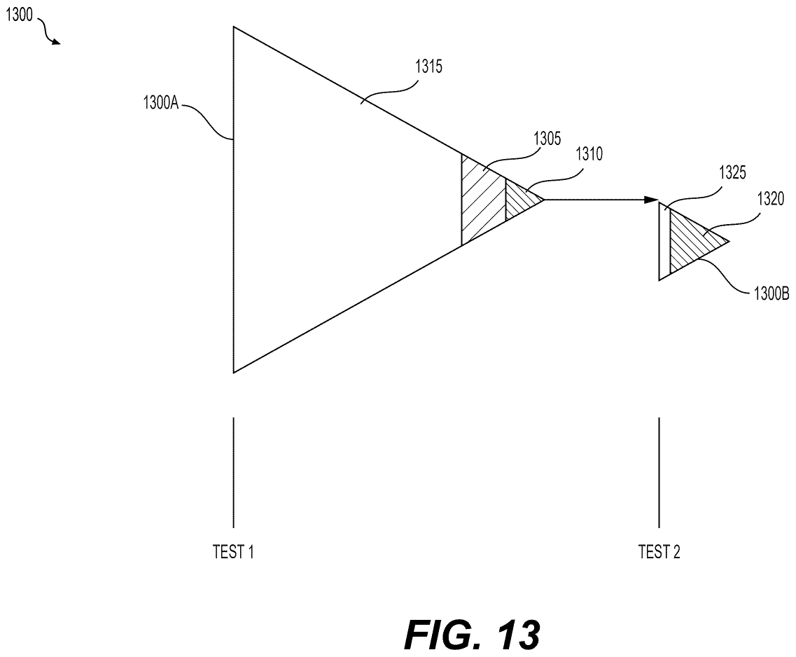

Referring now to FIG. 13, diagram 1300 illustrates a conceptual framework for improving MCED test performance through a two-stage testing strategy, according to one or more aspects of the present disclosure. More particularly, representation 1300A shows an initial test (Test 1) that identifies a small proportion of positives (represented by the narrowing cone), followed by representation 1300B, which shows a retest (Test 2) designed to refine and confirm those results. In this example, the dashed regions of the cones correspond to results that are either positive (region 1310, falling dashed lines) or indeterminate (region 1305, rising dashed lines). As illustrated, the bulk of the test results are negative, indicated by the blank space 1315 in the first cone. The positive results are then retested, with a portion of the positive results being confirmed positive (region 1320, falling dashed lines) and the rest being negative (region 1325). Diagram 1300 may correspond with the retesting system described herein. For instance, Test 1 administered at a first time (e.g., today) in a population of 10,000 individuals with a 1% test-positive rate, about 1,000 test positive, from which half are true cancers and half are false positives. Among those retested, roughly two-thirds of results turn negative, and very few cancers revert, suggesting that most retest negatives are indeed false positives. This implies that positive predictive value (PPV) could increase by virtue of retesting. Together, the framework illustrates how sequential testing can reduce false positives and enhance overall detection efficiency.

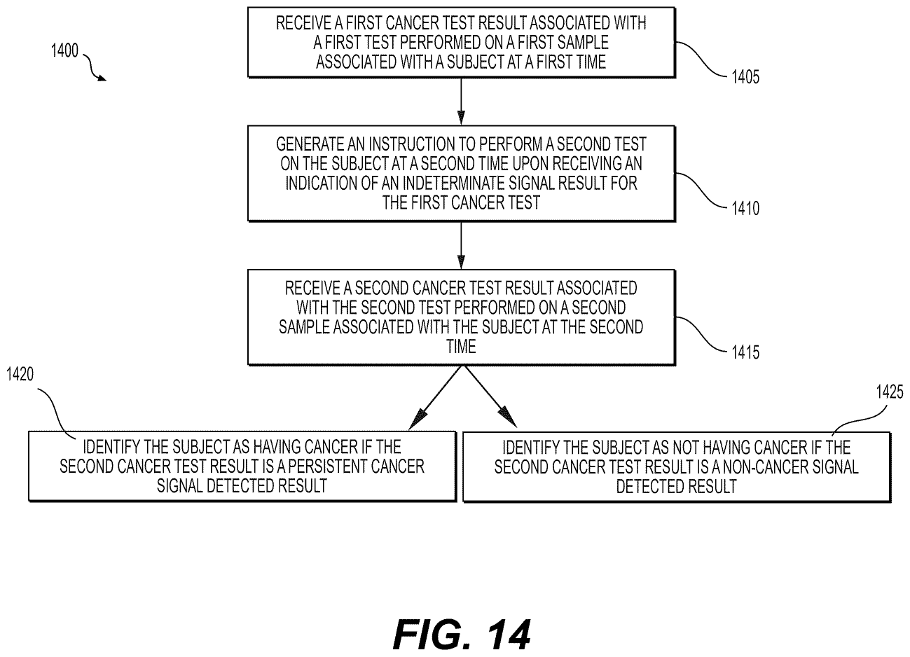

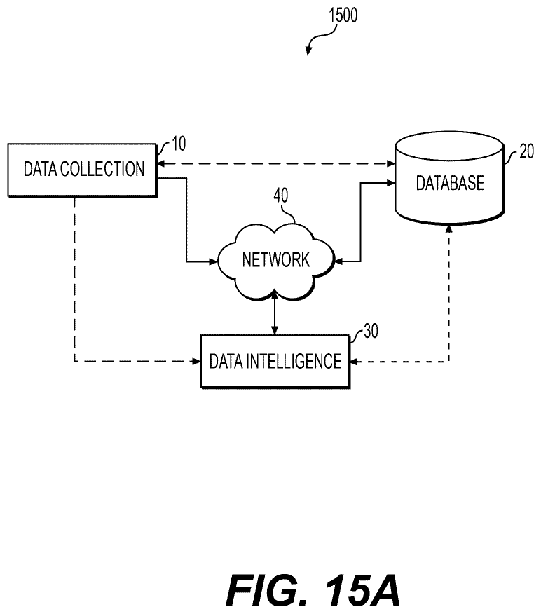

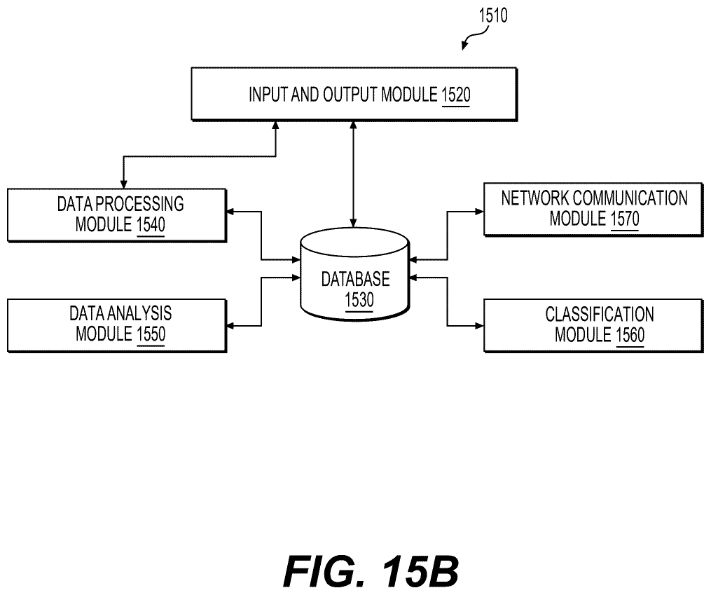

Referring now to FIG. 14, an exemplary workflow 1400 is described for improving the sensitivity and specificity of cancer screening in a population, according to one or more aspects of the present disclosure. Aspects of the exemplary workflow 1400 may be performed in accordance with some or all of the components described in FIGS. 15A and 15B.

At step 1405, the system may be configured to receive a first cancer test result associated with a first biological sample collected from a subject at a first time point. The first test may include an MCED assay, a cfDNA methylation-based assay (alone or in combination with other biomarkers such as protein, RNA, mutations, etc.), or another molecular diagnostic test configured to detect the presence or absence of a cancer-associated signal in the sample. The system may receive the first cancer test result via a network connection, an application programming interface (API), or a laboratory information management system (LIMS) following completion of the assay and corresponding data analysis.

In an aspect, the first cancer test result may be one of several categorical outputs generated by the test algorithm, including a cancer signal detected (CSD) result, an indeterminate signal result, or a no cancer signal detected NCSD result. In certain embodiments, the CSD result indicates that a probability that the first biological sample came from a subject with cancer exceeds a predefined detection threshold. The probability may be assessed by evaluating the measured molecular features, such as methylation patterns, fragmentomic signatures, or nucleic acid sequence alterations associated with the presence of cancer-derived material. The probability may be determined by a machine-learned classifier or other algorithmic interpretation of the measured molecular features. The NCSD result may indicate that the probability is below a defined limit of detection or falls within a range characteristic of non-cancer biological variation. The indeterminate signal result may represent a result that falls within an intermediate range, for example between a high threshold associated with a CSD result and low threshold associated with a NCSD result, near a decision boundary, where the confidence of cancer signal detection does not satisfy a predefined statistical criterion or threshold. In some examples, the indeterminate classification is generated when the computed probability of cancer signal presence lies between two boundary values (e.g., 0.4-0.6, 0.75-0.9, 0.85-0.95, etc.), thereby triggering subsequent analysis or retesting procedures. The received first test result is stored in an electronic data structure associated with the subject, and the system may also record metadata such as the collection time, sample type, assay batch, and processing parameters for use in subsequent computational workflows.

At step 1410, the system may generate, responsive to receiving the indeterminate signal results, an instruction to perform a second test on the subject at a second time after a predetermined time interval after the first time interval. In an aspect, the instruction to perform the second test may be output as a digital message, such as an alert, task order, or scheduling request transmitted to LIMS, a clinical trial management platform, or an electronic health record (EHR) system. The instruction may include details specifying the subject identifier, the type of test to be performed, the recommended retesting time window, and any relevant contextual information from the first test, such as cancer probability, test sensitivity, batch identifiers, or assay version.

In an aspect, upon classification of the first cancer test result as indeterminate, the system may evaluate predefined retesting criteria to determine the appropriate timing for a follow-up sample collection. The predetermined time interval may be defined as a fixed duration, e.g., one month, three months, six months, etc., regardless of the probability of cancer, or other clinical factors, or the time interval may be dynamically adjusted based on one or more subject-specific or assay-specific parameters. Such parameters may include the probability score associated with the indeterminate result, prior cancer screening history, cancer risk factors, estimated tumor fraction, statistical models of expected cfDNA dynamics, and the like. In certain implementations, the predetermined time interval may be derived from a predictive model trained on longitudinal cancer detection data. For example, the model may estimate the optimal retesting interval that maximizes diagnostic power while minimizing unnecessary testing burden. In one aspect, the model may use statistical simulations or survival-based optimization to predict how long it would take for a tumor fraction or methylation signal to rise such that the confidence in a subsequent result is expected to be definitive, based on historical growth patterns observed across similar cancer types.

In some aspects, the system may further incorporate a decision confidence threshold such that the instruction to retest is generated only if the indeterminate result satisfies certain criteria, e.g., when the classifier's output score is within a specific intermediate range or when the variance in assay signal exceeds a predefined tolerance. The generated instruction may be logged in a test management database or subject electronic health record along with timestamps, user identifiers, and audit metadata to ensure traceability and regulatory compliance.

At step 1415, the system may receive a second cancer test result associated with a second test performed on a second biological sample obtained from the same subject at a second time. The second test may be conducted after a predetermined time interval following the first test, as determined by the computer system or a clinical scheduling module, and may employ the same or a different assay platform as the initial test. For instance, in an aspect, the second test may include an MCED assay, a cfDNA methylation-based assay, or another molecular diagnostic test configured to detect the presence or absence of a cancer-associated signal in the sample. The system may receive the second cancer test result via a network connection, an API, or a LIMS following completion of the assay and corresponding data analysis. In an aspect, the second test may use the same or different threshold compared to the first test. In particular, the threshold (e.g., probability of cancer threshold) may be higher in the second test than in the first test. This may allow the first test to function as a wider net for detecting potential cancer-positive subjects (e.g., by reducing specificity to increase sensitivity) while the second net applies a finer filter to reduce false positives.

Similar to the first test result, the second test result may be one of a persistent CSD result or an NCSD result. In certain aspects, the persistent CSD result indicates that a probability of cancer (e.g., derived from evaluation of cancer-associated molecular signature identified in the first test) remains present in the second test at a consistent or elevated level, signifying biological persistence of the cancer signal over time. This persistence may be determined by comparing one or more quantitative parameters between the first and second test results, such as classifier probability scores, cfDNA tumor fraction estimates, methylation density metrics, fragment size distributions, and the like. A persistent CSD result may correspond to cases in which the computed cancer probability remains above a predefined detection threshold (e.g., ≥0.8) or does not decrease by more than a defined tolerance level across the two time points.

Conversely, an NCSD result for the second test may indicate that the probability of cancer in the second cancer has fallen below a given threshold, no detectable cancer-associated signal was observed, or that the previously observed indeterminate or weak signal has diminished below a level of concern or the assay's LoD. The system may interpret the NCSD outcome as evidence that the signal observed in the first test was transient or non-cancer in origin, thereby supporting a non-cancer classification for the subject. In some aspects, the system may also store the second test result alongside the first result within a subject-specific record, enabling longitudinal analysis and correlation of test results over time.

In some aspects, the received second test result may include additional metadata or intermediate output values from the assay pipeline, such as raw classifier logits, quality control metrics, or coverage statistics. The system may use these values to assess the reliability of the second test result and to determine whether the observed change in signal between the first and second test is statistically significant. For example, a machine learning model or temporal signal persistence algorithm may be applied to the pair of test results to quantify the likelihood that the observed pattern corresponds to true cancer progression versus assay noise. The processed second test result and associated comparison metrics may be stored in a database and made available for subsequent interpretation, report generation, or automated clinical decision support.

At step 1420, the system may identify the subject as having cancer when the second cancer test result is classified as a persistent CSD result. In an aspect, upon receiving the second cancer test result, the system may execute a classification algorithm that evaluates the temporal consistency, magnitude, and quality of the detected molecular features across the first and second test results. The system may compare cancer-associated signal metrics, such as cfDNA methylation value or density, tumor fraction, fragment size distribution, or sequence variant frequency, to determine whether the signal observed at the first time persists or increases at the second time. If the persistence criteria are satisfied, such as when the computed cancer probability score remains above a threshold or the difference between the assessment of the first test and the second test shows a non-decreasing trend between the two time points or varies by an amount that does not satisfy a threshold amount, the system may generate an output classification identifying the subject as likely having cancer.

In some aspects, the persistence determination may involve leveraging a model that quantifies similarity between representations of molecular features (e.g., feature vectors) derived from the two samples. For example, a correlation or distance metric may be computed between the two test outputs to assess concordance in feature space. The system may identify the signal as persistent when the computed correlation exceeds a predefined similarity threshold, thereby indicating that a cancer-associated molecular signature is present in both tests. In some embodiments, the system may apply statistical or machine learning models trained to distinguish persistent cancer signals from transient assay noise or biological variability.

In another aspect, the system may assign a diagnostic confidence score representing the probability that the subject truly has cancer based on the observed persistence pattern. This score may be computed, e.g., using a Bayesian or machine learning inference model, that incorporates prior test performance characteristics, including sensitivity, specificity, and expected false positive rates. When the confidence score exceeds a predefined diagnostic threshold (e.g., 80%, 90%, 95%, etc.), the processor may automatically classify the subject as positive for cancer. By incorporating both longitudinal data and algorithmic analysis, the computer system may improve the accuracy and reliability of cancer identification compared to single time-point testing.

In an aspect, the identification of the subject as having cancer may trigger one or more downstream actions by the system. For instance, the system may update the subject's record within a database to reflect a positive cancer classification, generate an automated report summarizing the test results, and transmit a notification or clinical recommendation to a healthcare provider or study investigator. The report may include supporting information, such as classifier scores, tumor fraction estimates, and persistence confidence values, to assist with clinical interpretation. In some implementations, the system may also record the identification event and the corresponding second test data in an audit log for traceability and compliance purposes.

At step 1425, the system may identify the subject as not having cancer when the second cancer test result is classified as an NCSD result. More particularly, upon receipt of the second cancer test result, the system may evaluate the molecular and statistical parameters associated with that result to confirm the absence of a detectable cancer-associated signal or the cancer-associated signal falling below a set value. The NCSD result may indicate that the molecular features measured in the second sample, e.g., such as cfDNA methylation values, densities, or profiles, fragmentomic patterns, or variant signal intensities, etc., fall below a predefined detection threshold or within a range consistent with background biological noise. In an aspect, the system may compare the quantitative features of the second test to those of the first test to verify that the previously observed indeterminate signal has not persisted or increased, thereby supporting the conclusion that the initial signal was transient or non-cancer in origin.

In some aspects, the system may perform one or more confirmatory analyses to validate the NCSD result before classification. For instance, the system may apply a temporal signal decay model or trend analysis to determine that cancer-associated molecular markers decreased or remained stable across the two test time points. The system may further apply a decision threshold derived from a probabilistic model or classifier score, such that if the computed cancer probability for the second test is less than a predetermined value (e.g., ≤0.2, 0.5, 0.75, 0.8, 0.9, etc.), the system may automatically identify the subject as not having cancer. In an aspect, the system may generate an output classification indicating a negative cancer determination and store this result in a subject-specific record within a secure database. In some aspects, the system may also generate a report or digital message confirming the NCSD outcome and may transmit it to a clinician, laboratory system, or EHR platform.

FIG. 15A depicts an exemplary system for sequential molecular testing and analysis. Exemplary system 1500 includes a data collection component 10, a database 20, and a data intelligence component 30, operably connected to each other via network 40. In certain aspects, one or more of these components may communicate locally or remotely through any suitable wired or wireless connection.

The data collection module 10 may correspond to one or more instruments, systems, or software interfaces configured to receive biological samples and/or molecular assay results obtained from a subject at two or more time points. The data collection module may be responsible for acquiring, digitizing, and formatting molecular data suitable for computational analysis. The collected data may be transmitted via network 40 to a database 20, which may store, e.g., sequential assay results, temporal identifiers, feature vectors, classifier weights, and associated subject metadata. The data intelligence module 30 may perform automated or semi-automated evaluations of incoming molecular data across multiple time points. In some aspects, the data intelligence module 30 may compare the first and second test results to determine persistence, disappearance, or evolution of cancer-associated features. The data intelligence module 30 may apply algorithms, correlation scoring, or machine learning models trained to identify temporal signal stability and classify each subject as likely cancer or non-cancer. Based on the outcomes of these analyses, the data intelligence module 30 may update subject classification status, trigger recommendations for follow-up testing, or store the analysis outcomes within the database 20 for further review. In certain aspects, operator review inputs or clinical overrides may also be processed and logged through this architecture.

FIG. 15B depicts an exemplary computer system 1510 that further enables acquisition, comparison, and classification of sequential molecular test data, as described in the present disclosure. Exemplary system 1510 achieves such functionalities by implementing, on one or more computer devices, user input and output (I/O) module 1520, memory or database 1530, data processing module 1540, data analysis module 1550, classification module 1560, network communication module 1570, and any other functional modules that may be needed for carrying out a particular task (e.g., a feature extraction module, a temporal comparison module, a reporting module, etc.), as will be described further below.

As disclosed herein, I/O module 1520 may further include an input sub-module, such as a keyboard, and an output sub-module, such as a display (e.g., a printer, a monitor, or a touchpad). In some aspects, all functionalities may be performed by one computer system. In some aspects, the functionalities are performed by more than one computer system. In some aspects, a user may use I/O module 1520 to manipulate data that is available either on a local device or may be obtained via a network connection from a remote service device or another user device. For example, I/O module 1520 may allow a user, e.g., via a keyboard, a mouse, or a touchpad, to initiate or perform data analysis via a graphical user interface (GUI). In some embodiments, a user may manipulate data via voice control. In some embodiments, user authentication may be required before a user is granted access to the data being requested. In some embodiments, I/O module 1520 may be used to manage various functional modules. For example, a user may request via I/O module 1520 input data while an existing data processing session is in progress. A user may do so by selecting a menu option or type in a command discretely without interrupting the existing process. In another example, a user may utilize I/O module 1520 to set various thresholds, configure signal persistence evaluation settings, and/or provide other instructions to computer system 1510 that dictate how data may be processed, compared, and classified. As disclosed herein, a user may use any type of input to direct and control data processing and analysis via I/O module 1520.

In an aspect, the I/O module 1520 may be configured to receive molecular assay results, metadata, and control parameters from one or more data collection instruments or laboratory systems. The input functionality may include options for uploading first and second test results associated with different time points, as well as parameter input mechanisms for defining test intervals, comparison thresholds, and classification rules. On the output side, the I/O module 1520 may present analytical results and classification outcomes, e.g., such as “persistent signal,” “transient signal,” or “no signal detected,” to a user via a graphical interface or a secure electronic dashboard. The I/O module 1520 may also enable authorized operator review, allowing users to validate, annotate, or override automated classifications when required.

In some embodiments, system 1510 further comprises a memory or database 1530. In some embodiments, database 1530 comprises a local database that may be accessed via I/O module 1520. In some embodiments, database 1530 comprises a remote database that may be accessed by I/O module 1520 via a network connection. In some embodiments, database 1530 is a local database that stores data retrieved from another device (e.g., a user device or a server). In some embodiments, memory or database 1530 may store data retrieved in real-time from internet searches. In some embodiments, database 1530 may send data to and receive data from one or more of the other functional modules, including, but not limited to, a data collection module (not shown), data processing module 1540, data analysis module 1550, classification module 1560, network communication module 1570, and other analytical components involved in sequential test comparison.

The database 1530 may function as the primary data repository and coordination hub for the automated sequential molecular testing framework. The database 1530 may be configured to store a wide range of data artifacts necessary for both real-time decision-making and retrospective review. This may include raw and processed molecular data from multiple test time points, extracted biomarker features, temporal identifiers, correlation matrices, and algorithmic outputs. The database 1530 may also store classification models, historical training data, predefined persistence thresholds, and clinical metadata associated with each subject or cohort. The database may serve as the central coordination hub for communication between the various analytical modules, ensuring data consistency and traceability across the sequential testing workflow.

In some aspects, database 1530 may be a database local to the other functional modules. In some embodiments, database 1530 may be a remote database that may be accessed by the other functional modules via wired or wireless network connection (e.g., via network communication module 1570). In some aspects, database 1530 may include a local portion and a remote portion.

System 1510 may comprise a data processing module 1540. Data processing module 1540 may receive data from I/O module 1520 or database 1530. In some embodiments, the data processing module 1540 may perform initial parsing, normalization, and formatting of molecular data received from the laboratory or data collection instruments. For instance, upon receiving assay result files, the data processing module 1540 may extract quantitative biomarker signals, normalize them according to internal controls, and generate standardized data structures for downstream temporal comparison. The data processing module 1540 may also extract derived metrics, such as feature stability indicators or signal intensity measures, and prepare them for further computational analysis.

The data analysis module 1550 may operate as the computational engine for temporal and statistical evaluation within the automated sequential testing workflow. Upon receiving processed molecular features from the data processing module 1540, the data analysis module 1550 may compare molecular signals between two or more time points to assess persistence, disappearance, or significant change. The data analysis module 1550 may compute similarity metrics, correlation coefficients, or other measures of temporal concordance and apply machine-learning algorithms trained to distinguish true positive cancer-associated signals from transient or non-cancer patterns.

In some aspects, system 1510 comprises a classification module 1560, which may embody a “machine-learning model” or “trained classifier.” As used herein, a “machine-learning model” or “trained classifier” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration. In some aspects, the machine-learning model may be trained on a combination of real and synthetic molecular test data obtained from subjects with known cancer outcomes.

The execution of the machine-learning model may include deployment of one or more machine-learning techniques, such as random forest, logistic regression, support vector machines, gradient boosting, or deep neural networks configured to evaluate temporal data patterns. Supervised, semi-supervised, and/or unsupervised training may be employed.

As disclosed herein, network communication module 1570 may be used to facilitate communications between a user device, one or more databases, and any other suitable system or device through a wired or wireless network connection. Any communication protocol/device may be used, including, without limitation, a modem, an Ethernet connection, a network card (wireless or wired), an infrared communication device, a wireless communication device, and/or a chipset (such as a Bluetooth™ device, an 802.11 device, a WiFi device, a WiMax device, cellular communication facilities, etc.), a near-field communication (NFC), a Zigbee communication, a radio frequency (RF) or radio-frequency identification (RFID) communication, a PLC protocol, a 3G/4G/5G/LTE based communication, and/or the like. For example, a user device having a user interface platform for initiating or reviewing testing of patient samples may communicate with another user device with the same platform, a regular user device without the same platform (e.g., a regular smartphone), a remote server, a physical device of a remote IoT local network, a wearable device, a user device communicably connected to a remote server, etc.

The functional modules described herein are provided by way of example. It will be understood that different functional modules may be combined to create different utilities. It will also be understood that additional functional modules or sub-modules may be created to implement a certain utility.

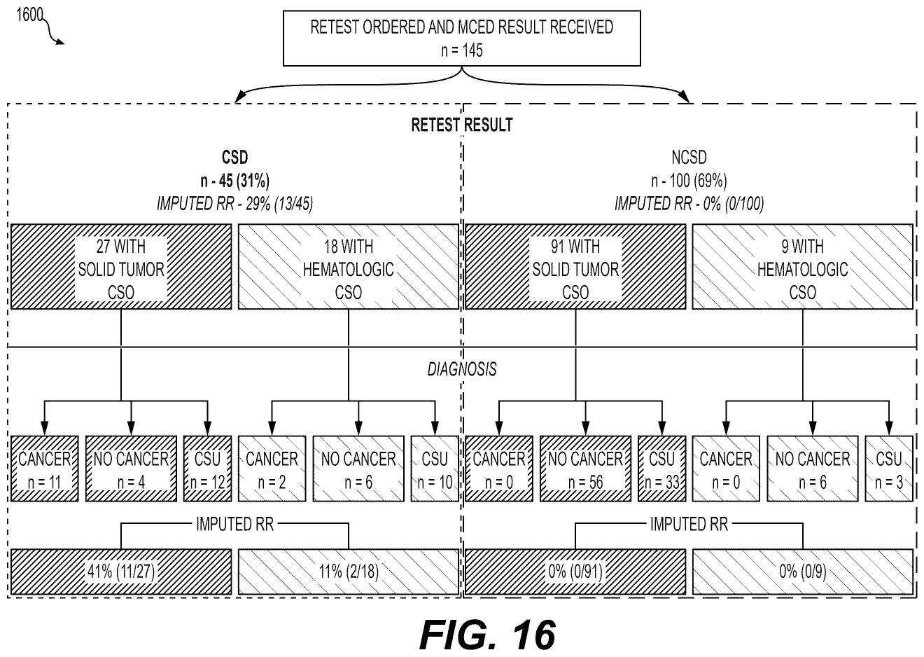

In an aspect, a retesting framework for MCED through sequential testing is disclosed that highlights a first approach of retesting individuals who initially test positive at a later time to clarify the persistence of a cancer signal. In this approach, retesting is performed at a 3 to 6-month interval after the initial test. This approach aims to determine whether an initial positive result represents a true positive cancer signal or a transient signal that may not require further intervention. More particularly, referring now to FIG. 16, diagram 1600 summarizes clinical outcomes for 145 individuals who initially received a cancer signal detected (CSD) result from an MCED test and subsequently underwent retesting. Of these subjects, 69% received a “no cancer signal detected” (NCSD) result upon retesting. This group was found to have a residual risk of 0%, indicating a low likelihood of cancer following a negative retest. Conversely, 31% of individuals with a persistent CSD on the retest exhibited a higher likelihood of an actual cancer diagnosis, with an imputed residual risk of 29%. These findings reinforce the utility of retesting to help stratify patients based on persistent versus transient cancer signals, even with a longer follow-up time period (3-6 months) than that proposed by this disclosure (e.g., as short as 1-8 weeks).

Table 1 below presents both observed and imputed residual risks of cancer among 145 individuals who underwent retesting following an initial MCED test showing a CSD result. Table 1 distinguishes between solid tumor and hematologic cancer signal origins (CSOs) and compares risk estimates before and after retesting. Before retest, the overall observed residual risk was 14%, with similar rates for solid tumor and hematologic CSO. After retesting, the risk patterns diverged, with individuals with a persistent CSD result on retest having an observed residual risk of 23%, while those whose retest result converted to NCSD had an observed residual risk of 0. This data indicates that a persistent CSD retest substantially increases the likelihood of a true cancer diagnosis, whereas a negative NCSD retest strongly correlates with the absence of current cancer.

TABLE 1

| | | | | |
|---|---|---|---|---|
| | Clinical | Cancer | Observed | Imputed |
| | Status | Diagnosis | Residual | Residual |
| | Available, | Confirmed, | Risk | Risk, |
| Total | N | n | n/N | n/Total |

Residual Risk Before Retest by CSO Type

| | | | | | | | |
|---|---|---|---|---|---|---|---|
| First test, | 145 | 93 | 13 | 13/93 | (14) | 12/145 | (9) |
| All CSOs | | | | | | | |
| Solid Tumor | 120 | 73 | 11 | 11/73 | (15) | 11/120 | (9) |
| CSO | | | | | | | |
| Hematologic | 25 | 14 | 2 | 2/14 | (14) | 2/25 | (8) |

CSO

Residual Risk After No Cancer Confirmation

| | | | | | | | |
|---|---|---|---|---|---|---|---|
| CSD on | 45 | 23 | 13 | 13/23 | (57) | 13/45 | (29) |
| Retest | | | | | | | |
| Solid Tumor | 27 | 15 | 11 | 11/15 | (73 | 11/27 | (41) |
| CSO | | | | | | | |
| Hematologic | 18 | 8 | 2 | 2/8 | (25) | 2/18 | (11) |
| CSO | | | | | | | |
| NCSD on | 100 | 64 | 0 | 0/64 | (0) | 0/100 | (0) |

Retest

Table 2 below provides detailed information on the CSO, clinical characteristics, and cancer histories of the 13 patients diagnosed with cancer following retesting. The table shows that detected cancers spanned multiple tissue types, including, breast, head and neck (notably HPV-positive cancers), lung, lymphoid lineages (Hodgkin and Non-Hodgkin lymphomas), pancreas, gallbladder, neuroendocrine tumors, and stomach/esophagus. Cancer stages ranged from early (Stage I) to advanced (Stave IV or recurrent), indicating diverse disease progression at diagnosis. This data indicates that a CSD retest strategy is equally viable across a wide variety of cancer types, stages, and patient histories.

TABLE 2

| | | Diagnosed | | | |
|---|---|---|---|---|---|
| | | Cancer | | | Prior |
| | | type | Cancer | | Cancer |
| Retest CSO | Test 1 CSO | (AJCC) | Stage | Histologic Type | History |
| Breast | Breast | Breast | IV | Adenocarcinoma, | No |
| | | | | NDS | |
| Breast | Prostate | Major | IV | Infiltrating | No |
| | | Salivary | | duct carcinoma, | |
| | | Glands | | NOS | |
| Head and | Breast | Vagina | IV | Squamous cell | Yes |
| Neck | | | | carcinoma, | |
| | | | | NOS | |
| Head and | Head and | HPV+ | I | Squamous cell | No |
| Neck | Neck | OPC | | carcinoma, | |
| | | | | HPV+ | |
| Head and | Head and | HPV+ | Distant | Squamous cell | Yes |
| Neck | Neck | OPC | Recurrent | carcinoma, | |
| | | | | HPV+ | |
| Head and | Head and | HPV+ | IV | Squamous cell | No |
| Neck | Neck | OPC | | carcinoma, | |
| | | | | HPV+ | |
| Lung | Lung | Lung | III | Adenocarcinoma, | No |
| | | | | NDS | |
| Lymphoid | Lymphoid | Hodgkin | III | Follicular | Yes |
| Lineage | Lineage | and NHL | | lymphoma, | |
| | | | | NOS | |
| Lymphoid | Lymphoid | Hodgkin | NA | Malignant | Yes |
| Lineage | Lineage | and NHL | | lymphoma, | |
| | | | | non-Hodgkin, | |
| | | | | NOS | |
| Neuroendocrine | Neuroendocrine | Testin | NA | Germinoma | No |
| Pancreas, | Pancreas, | Breast | Distant | Infiltrating | Yes, |
| Gallbladder | Gallbladder | | Recurrent | duct carcinoma, | Breast |
| | | | | NOS | |
| Pancreas, | Pancreas, | Missing | IV | Adenocarcinoma, | Yes |
| Gallbladder | Gallbladder | | | NDS | |
| Pancreas, | Stomach, | Gallbladder | NA | Adenocarcinoma, | No |
| Gallbladder | Esophagus | | | NDS | |

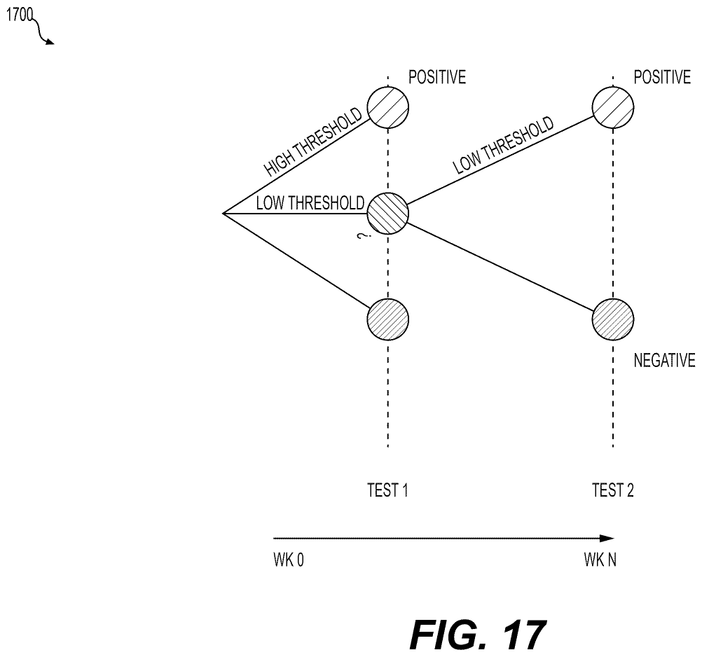

Referring now to FIG. 17, diagram 1700 depicts a flowchart for a sequential testing algorithm intended to categorize individuals based on their initial cancer signal and inform a subsequent retesting strategy. The timeline spans two primary test points, labeled as Test 1 and Test 2, with each pathway determined by initial and follow-up test results and threshold criteria. In an aspect, individuals receiving the first test may be sorted into three categories based on the classifier's thresholds: low signal (narrow rising diagonal lines), intermediate signal (falling diagonal lines), and high signal (wide diagonal rising lines). Individuals who fall below a low threshold are likely deemed to have negligible cancer signal and are not considered for retesting as the predicted probability of the individual having cancer (i.e. p (cancer)) is low. These individuals are classified as “negative” after the first test, as there is minimal benefit in further assessment given the low probability of cancer. Individuals who fall in the intermediate category have a cancer signal that falls within a high-risk “intermediate” zone, which lies between a low-specificity p (cancer) threshold (e.g., to accommodate a 95%, 97%, 98.5%, etc. specificity) and a higher-specificity p (cancer) threshold (e.g., to accommodate a 99%, 99.5%, 99.6%, 99.7%, etc. threshold). This intermediate range captures cases in which there is some indication of cancer signal, but the signal is not strong enough to confirm a positive result confidently. These individuals may be flagged for retesting at a subsequent time (e.g., week N, where N could be between 2 and 24 weeks, preferably between 3 and 8 weeks). Upon retesting, their results may either decay to a “negative” classification (e.g., indicating a likely false positive in the initial test) or persist as “positive,” which would prompt further action for a cancer workup. For the last category, individuals who exceed the high p (cancer) threshold may be strongly suspected to have a true cancer signal. These cases may immediately be classified as “positive” and are recommended for prompt workup, bypassing the need for retesting. Their high signal strength suggests a high likelihood of cancer, so any delay in follow-up may be disadvantageous. Although thresholds based on 98.5% specificity and 99.5% specificity are referenced herein, any suitable specificity threshold may be used.

In an aspect, the focus of the retesting procedure described in this disclosure may primarily be on the intermediate group, where a second test result provides further clarity on the cancer signal's persistence. A positive result on the second test may indicate that the individual should be classified as positive, reinforcing the likelihood of true cancer presence. Conversely, a negative result on the second test implies that the initial result may have been a transient or false positive signal, allowing the algorithm to classify the individual as overall negative. This approach enables a more nuanced categorization. For example, the classifier may be trained as two classifiers with dynamically adjusted sensitivities across two tests rather than as a single point-time-in test.

The retesting strategy observes whether an initial cancer signal persists or decays at a second time point, classifying persistent signals as potentially true positives and decayed signals as likely false positives. However, in an aspect, information from both the initial test and the follow-up test may be leveraged to improve classifier performance by allowing the classifier to “condition” on the initial test's signal in its analysis of the follow-up test results. In an aspect, the result of the first test informs the interpretation of the second. For instance, by conditioning on the initial cancer signal strength, pattern, biomarker profile, etc., the classifier may better assess whether a second, possibly weaker, signal still suggests cancer. For example, if an individual's first test shows a moderately high cancer signal, the classifier may be trained to consider a slightly lower signal in the second test as significant, interpreting it as confirmation of a cancer-related signal rather than a transient false positive. Conversely, if the first test yields a borderline result and the second test shows a complete decay, this conditional analysis may reinforce the likelihood of a false positive, reducing unnecessary follow-up.

In an aspect, the foregoing approach may leverage one, two, or more classifiers. In the case of one classifier, the difference between the application of the test at the first time point and application of the test at the second time point may differ only in the p (cancer) threshold associated with a target specificity (i.e., a lower p (cancer) associated with a lower target specificity for the first application and a higher p (cancer) associated with a high target specificity for the second application). The remainder of the operation of the classifier is the same. In another aspect utilizing one classifier, the difference in the application of the test at the first time point and application of the test at the second time point may different only in that the application of the test at the first time point uses multiple thresholds (e.g., a high threshold corresponding to a high specificity that results in a CSD result for samples that exceed the high threshold, and a low threshold corresponding to a lower specificity that results in a NCSD results for samples that come below the low threshold) while the application of the test at the second time point only uses a single threshold (which may be equal to the low threshold, or between the low threshold and the high threshold).

In the case of multiple classifiers, the first classifier may be trained to identify potential cancer signals at an initial screening point. It may be configured to operate with a broader sensitivity threshold, capturing possible positive cases but without overly strict specificity. This first classifier may be configured to flag individuals who have a detectable cancer signal for further evaluation. The second classifier may be optimized to interpret results conditionally, based on the information from the first test. By “conditioning” on the first test's results, the second classifier may essentially function as a “refining” classifier, improving the ability to distinguish true positives from false positives. Additionally or alternatively, additional classifiers may be used conditioned on the specific results of the first classifier (e.g., the cancer signal result).

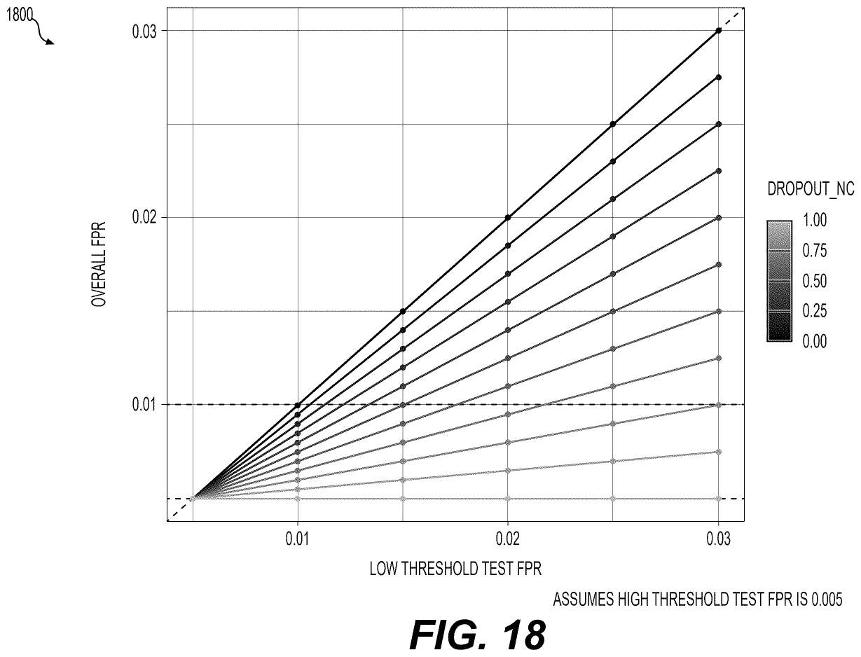

FIGS. 18-25, and their associated disclosure, provide a description of modeling and other experiments used to demonstrate the feasibility and validate the retesting procedure as described herein.

Referring now to FIG. 18, graph 1800 illustrates how the overall false positive rate (FPR) behaves in a sequential testing framework for multi-cancer detecting, according to one or more aspects of the present disclosure. In this experiment, the difference between the two tests is exclusively the p (cancer) threshold. Equation 1 below models the overall FPR (f) as a function of the FPRs of the low-threshold test (f_{low}) and high-threshold test (f_{high}), as well as the dropout rate of non-cancer individuals (π_{nc}), e.g., the proportion of individuals who do not return for the second test. The plotted curves demonstrate that as the dropout rate of non-cancer individuals decreases (i.e., more non-cancer individuals are given the high-threshold confirmatory test), the overall FPR increases, reflecting the fact that it is advantageous to use the low-threshold test to filter out subjects with low probability of cancer. Conversely, when dropout rates are high, the overall FPR remains skews lower, showing that the p (cancer) for the low-threshold test does not need to exclude all non-cancer individuals from the confirmatory test to meaningfully reduce the overall FPR.

$$f = {f}_{high} + ( 1 - {π}_{n c} ) ( {f}_{low} - {f}_{high} ) \qquad Equation 1$$

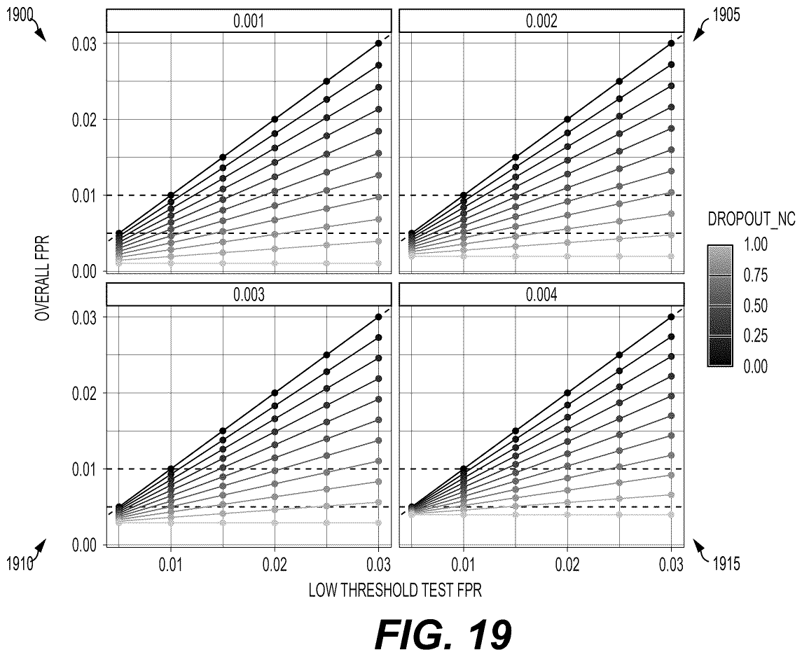

Referring now to FIG. 19, graphs 1900-1915 collectively illustrate how the FPR behaves in a sequential testing framework across multiple settings for the high-threshold test's false positive rate, according to one or more aspects of the present disclosure. In this experiment, the difference between the two tests is exclusively the p (cancer) threshold. Each graph 1900-1915 represents a different target FPR for the high-threshold, confirmatory test, ranging from 0.001 to 0.004 (corresponding to specificities of 99.9-99.6, respectively). Within each graph 1900-1915, the x-axis shows the FPR of the initial low-threshold test, while the y-axis shows the resulting overall FPR of the combined sequential testing process. Each line represents different dropout rates of non-cancer individuals, ranging from 0 (no dropout) to 1 (complete dropout). The graphs 1900-1915 demonstrate that as dropout among non-cancer participants decreases (i.e., more non-cancer individuals are given the high-threshold confirmatory test), the overall FPR rises toward the FPR of the low-threshold test, confirming that the FPR of the low-threshold test is the ceiling of the overall FPR and further confirming that the overall FPR benefits from improved FPR of the initial test. Conversely, when dropout is high, the overall FPR benefits, indicating that sequential testing may effectively reduce false positives relative to single-test systems.

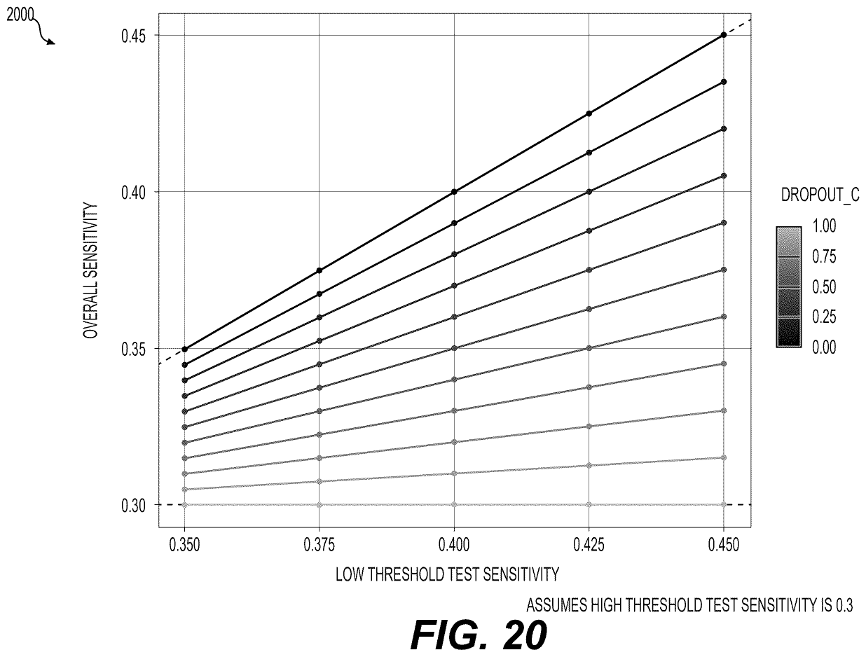

Referring now to FIG. 20, graph 2000 illustrates how overall sensitivity behaves in a sequential testing framework for multi-cancer detection, according to one or more aspects of the present disclosure. Equation 2 below defines overall sensitivity(S) as a function of the sensitivities of the low-threshold test (S_{low}) and high-threshold confirmatory test (S_{high}), as well as the dropout rate of cancer-positive individuals (π_{c}). The curves in the graph 2000 demonstrate that as dropout among cancer-positive individuals increase, overall sensitivity declines, since missed follow-up tests lead to undetected cancers that would otherwise be confirmed. Conversely, when dropout is minimal, the overall sensitivity approaches that of the low-threshold test, reflecting the benefit of retesting all positive cases. Graph 2000 therefore shows that sequential testing may maintain or improve sensitivity relative to a single, high-specificity test, provided that individuals with cancer complete both testing stages.

$$S = {S}_{high} + ( 1 - {π}_{c} ) ( {S}_{low} - {S}_{high} ) \qquad Equation 2$$

Table 3 provided below demonstrates how adjusting classifier thresholds to operate at progressively lower specificities (from 99.5% to 97.0%) affects multiple performance metrics across both testing stages. As specificity decreases, overall and stage-specific sensitivities increase, reflecting that a more permissive threshold allows the system to capture a greater proportion of true cancer signals. Correspondingly, the “High Signal” columns represent the subset of cases showing strong cancer-associated signals, which maintain high values even as specificity relaxes. The “Yield” metric, which represents the proportion of true cancers identified in the screened population, rises steadily as specificity decreases, improving overall detection efficiency. However, this gain is balanced by a decline in the positive predictive value (PPV), indicating that a greater fraction of positive results may represent false positives. The “Number Needed to Screen” (NNS) decreases as specificity relaxes, meaning fewer individuals need to be screened to detect one true cancer, which suggests higher population-level efficiency under sequential testing conditions.

TABLE 3

| | | High | | | |
|---|---|---|---|---|---|
| Target | Sensitivity | Signal | | | |
| Specificity | Stage All | Stage All | Yield | NNS | PPV |
| 99.5 | 38.0 | 72.7 | 0.62 | 161 | 60.5 |
| | (38.0, | (72.9, | (0.62, | | (59.7, |
| | 38.1, | 72.8, | 0.62, | | 59.6, |
| | 38.0) | 72.4) | 0.61) | | 62.3) |
| 98.5 | 43.6 | 78.8 | 0.70 | 143 | 32.2 |
| | (43.7, | (79.0, | (0.69, | | (32.8, |
| | 43.5, | 78.7, | 0.70, | | 32.3, |
| | 43.7) | 78.8) | 0.70) | | 31.6) |
| 98.0 | 45.1 | 80.1 | 0.72 | 139 | 26.5 |
| | (45.1, | (79.8, | (0.71, | | (26.9, |
| | 45.2, | 80.3, | 0.72, | | 27.0, |
| | 46.4) | 80.3) | 0.72) | | 25.6) |
| 97.5 | 46.3 | 81.1 | 0.73 | 137 | 23.2 |
| | (46.3, | (81.0, | (0.72, | | (23.6, |
| | 46.2, | 81.4, | 0.73, | | 23.9, |
| | 46.4) | 81.1) | 0.73) | | 22.1) |
| 97.0 | 47.2 | 81.9 | 0.74 | 135 | 20.7 |
| | (47.2, | (81.7, | (0.73, | | (21.0, |
| | 47.2, | 82.2, | 0.74, | | 20.3, |
| | 47.1) | 81.9) | 0.74) | | 19.5 |

Analysis of the foregoing metrics indicates that in a prototype model, the most influential unknown parameters affecting performance are the dropout rates of both cancer and non-cancer individuals who receive intermediate or indeterminate results. Based on model behavior, a low-threshold test specificity of approximately 98% appears to be a practical and effective target, balancing sensitivity and specificity. Maintaining the confirmatory high-threshold test specificity above 99.5% may enable the overall sequential testing procedure to preserve very high specificity while improving overall detection performance.

In an aspect, commercial testing operations and clinical surveillance programs may be leveraged to gather retest data efficiently and further tune the appropriate target specificity for the low threshold and high threshold tests. More particularly, in an aspect, given that approximately 1% of tests are expected to yield positive results, all positive cases may be retested within about one month of the initial test. To strengthen model calibration, additional retests may be conducted on borderline or high-scoring negative samples (e.g., those testing positive under a 98% specificity threshold but negative under a stricter 99.5% threshold). In an aspect, a follow-up time window may be maintained to confirm outcomes and distinguish between false positives and true positives, promoting accurate longitudinal performance assessment.

In one non-limiting experimental design, approximately 1,600 individuals with positive tests may be retested for evaluation purposes or 3,200 individuals when both evaluation and training are required, providing roughly a 50/50 training-test data split. Assuming a positive predictive value (PPV) of around 25%, approximately 400 of the retested individuals are expected to be true positives, while about 1,200 would represent false positives. Scaling these estimates to population-level screening, achieving a 1% cancer signal detection rate would require screening around 160,000 individuals to compile an appropriate data set to validate the retesting procedure, whereas a 2.5% detection rate would require screening about 64,000 individuals.

Referring now to Table 4 below, a paired study design is presented that is used to demonstrate the non-inferiority of the performance of the sequential MCED test compared to a single point-in-time MCED test. Specifically, Table 4 illustrates how outcomes from the first MCED test are compared to results from the overall sequential testing framework for individuals confirmed to be non-cancer. Each cell in the matrix (X₁₁, X₁₀, X₀₁, X₀₀) represents the count of individuals classified as positive or negative at each testing stage. The total number of observations, N, equals the sum of all four outcome categories. The model assumes that the vector of observed outcomes, X, follows a multinomial distribution with parameters N and p=(p₀₀, p₀₁, p₁₀, p₁₁), where each pᵢⱼ corresponds to the probability of a specific test result combination. This framework allows estimation of joint probabilities and dependencies between first-test and sequential-test outcomes.

TABLE 4

| | Positive | Negative |
|---|---|---|
| Positive | X₁₁ | X₁₀ |
| Negative | X₀₁ | X₀₀ |

In an aspect, differences in FPRs between two sequential tests may be expressed using multinomial probabilities. The framework models the joint outcomes of both tests as a multinomial distribution with probabilities p₀₀, p₀₁, p₁₀, and p₁₁, representing all possible combinations of positive and negative classifications. The marginal FPR for the first test (π₁) is defined as p₁₀+p₁₁, while the marginal FPR for the second test (π₂) is p₀₁+p₁₁. The difference in FPRs between the two tests (π₁−π₂) therefore simplifies to p₀₁−p₁₀, capturing the net change in misclassifications between stages.

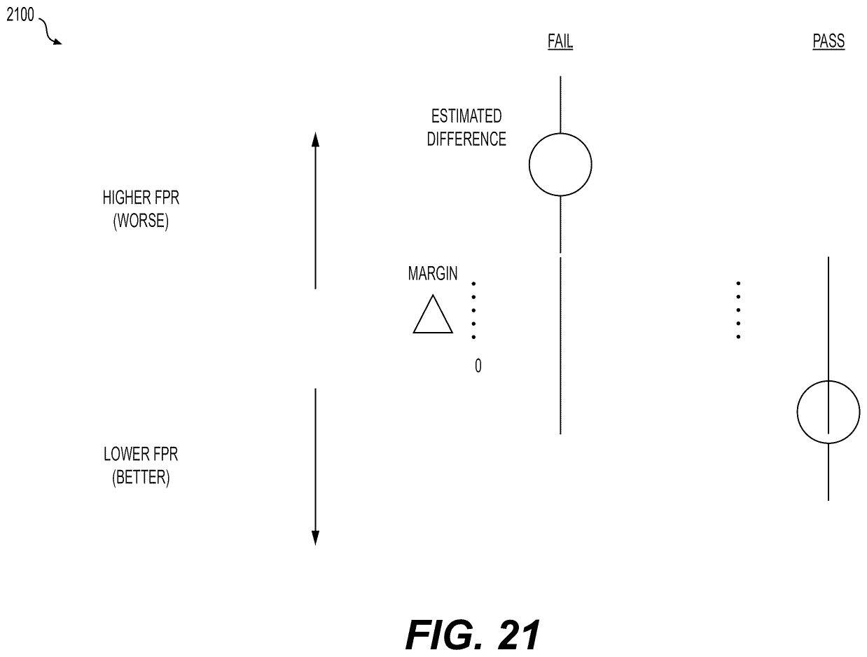

Referring now to FIG. 21, diagram 2100 illustrates the conceptual workflow for a power simulation used to evaluate differences in FPR between sequential tests, according to one or more aspects of the present disclosure. In an aspect, the process may begin by defining simulation parameters such as the FPRs of the first and second tests, their correlation, and the total sample size, according to one or more aspects of the present disclosure. A predefined margin of acceptable difference is then specified as the test parameter. Using these values, multinomial probabilities are derived to simulate a 2×2 contingency table representing paired outcomes from both tests. The upper bound of the 95% confidence interval for the difference in FPR is calculated, and the test outcome is determined by whether this bound falls below the specified margin. The accompanying diagram visualizes this logic: if the estimated difference and its confidence interval exceed the margin (indicating a higher FPR), the result “fails,” while if the upper bound lies below the margin (indicating a lower or improved FPR), the result “passes.” This approach quantifies the statistical power to detect meaningful improvements in FPR performance under different design assumptions.

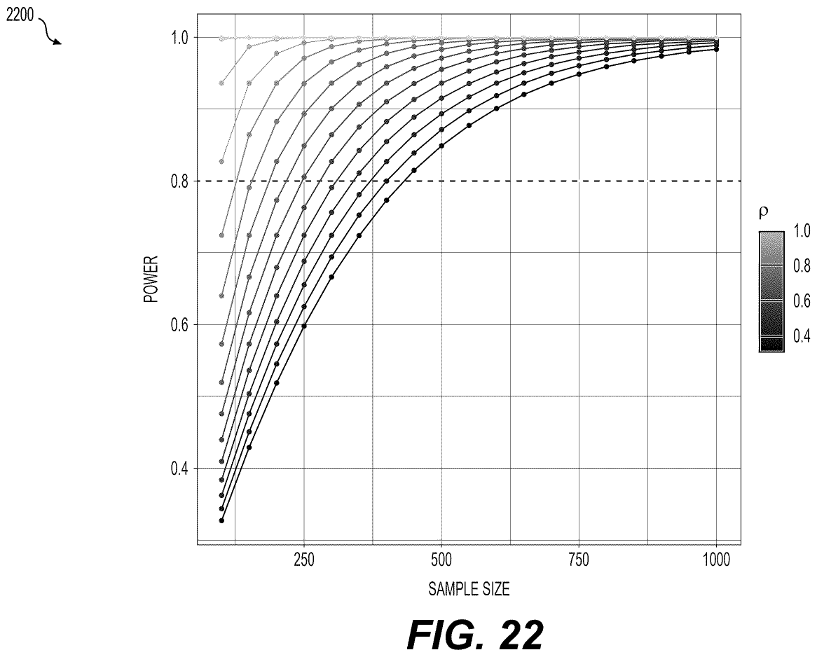

Turning now to FIG. 22, graph 2200 shows the results of a power simulation evaluating the ability to detect a difference in FPR between two sequential tests using an analytic calculation under a normal approximation, according to one or more aspects of the present disclosure. The x-axis represents sample size, while the y-axis represents statistical power, or the probability of correctly identifying a true difference in FPR. Each curve corresponds to a different correlation coefficient (p) between the outcomes of the two tests, ranging from 0.4 to 1.0. Higher correlation values indicate more similar test results and lead to greater statistical power at smaller sample sizes, while lower correlations require larger sample sizes to achieve equivalent power. The dashed horizontal line marks the conventional 80% power threshold. The analysis assumes both tests have an FPR of 0.5%, with a 1% margin used to define the difference of interest. Overall, the plot demonstrates that moderate-to-high correlation between sequential test outcomes substantially improves study efficiency, allowing reliable detection of FPR differences with fewer samples.

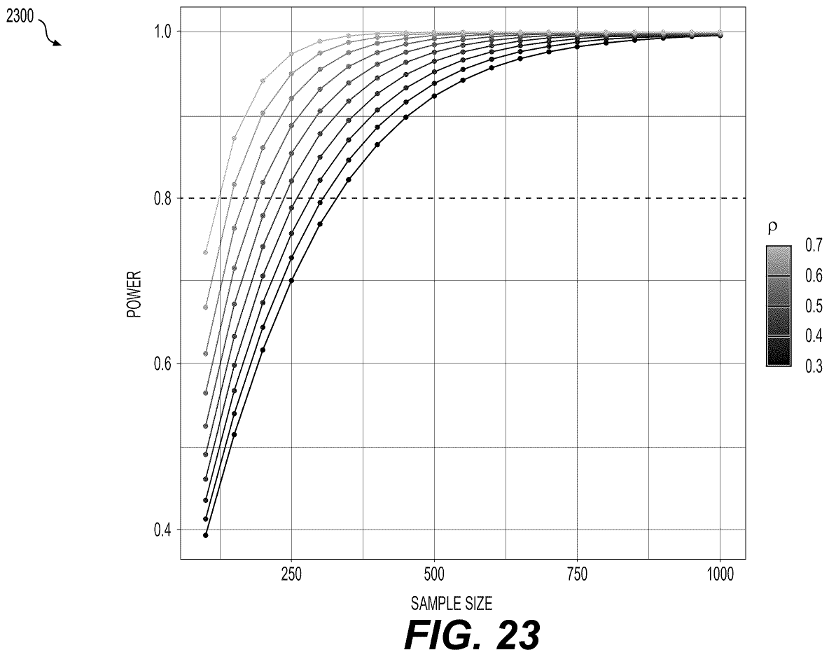

Turning now to FIG. 23, graph 2300 shows the results of a power simulation evaluating the ability to detect a difference in true positive rate (TPR), or sensitivity, between two sequential tests using an analytic calculation under a normal approximation, according to one or more aspects of the present disclosure. The x-axis represents the sample size, and the y-axis represents statistical power. Each curve corresponds to a different correlation coefficient (p) between the outcomes of the two tests, ranging from 0.3 to 0.7. Higher correlations yield greater power at smaller sample sizes, while lower correlations require more samples to achieve equivalent power. The simulation assumes the first test has a TPR of 20% and the second test has a TPR of 30%, representing a 10% improvement in detection sensitivity, with a 3% margin of difference used for hypothesis testing. The dashed horizontal line indicates the 80% power threshold. The results show that moderate correlation between test outcomes substantially increases the efficiency of detecting meaningful improvements in sensitivity, supporting feasible study designs used for validating sequential testing performance in accordance with this disclosure.

Table 5 below represents a paired study design framework for evaluating sequential MCED testing. More particularly, Table 5 represents possible outcomes between a first MCED test and the overall sequential test, where X₁₁, X₀₁, and X₀₀ denote observed counts of positive or negative results across test stages. By design, X₁₀=0 because samples testing positive at the high threshold are not retested. The total sample size N equals the sum of all observed outcomes. The parameter β is modeled using a Beta distribution, β˜Beta (a,b), while X₀₁ follows a Binomial distribution, X₀₁˜Binomial (N, β). Here, β represents the difference in a performance metric (e.g., sensitivity) between the two tests, and β\hat{ } simplifies to X₀₁/N. This structure enables probabilistic modeling of paired test performance, providing a statistical framework for quantifying differences in detection sensitivity.

TABLE 5

| | Positive | Negative |
|---|---|---|
| Positive | X₁₁ | X₁₀ = 0 |
| Negative | X₀₁ | X₀₀ |

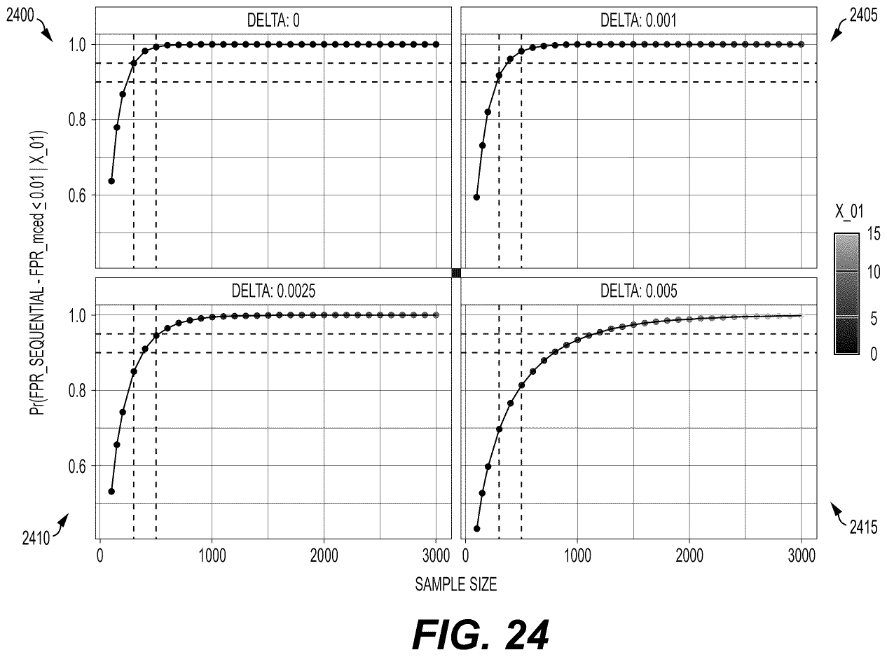

Referring now to FIG. 24, graphs 2400-2415 show the results of a paired study design simulation assessing the FPR for a sequential testing system compared to an initial MCED test, according to one or more aspects of the present disclosure. Graphs 2400-2415 represent different assumed true differences (“deltas”) between the FPRs of the two tests, ranging from 0 to 0.0005. The x-axis shows the total sample size, while the y-axis indicates the posterior probability that the sequential test's FPR is less than or equal to that of the initial test by a margin of 1%. The scale represents observed counts of discordant positive results (X₀₁) and the dashed horizontal lines denote 90% and 95% posterior probability thresholds. Across all scenarios, the posterior probability of demonstrating a lower FPR for the sequential test increases rapidly with sample size and approaches near certainty beyond approximately 500 to 1,000 samples, depending on the true underlying difference. These results suggest that even modestly sized studies may provide strong statistical evidence that a sequential testing approach maintains or improves false positive performance relative to a single-test framework.

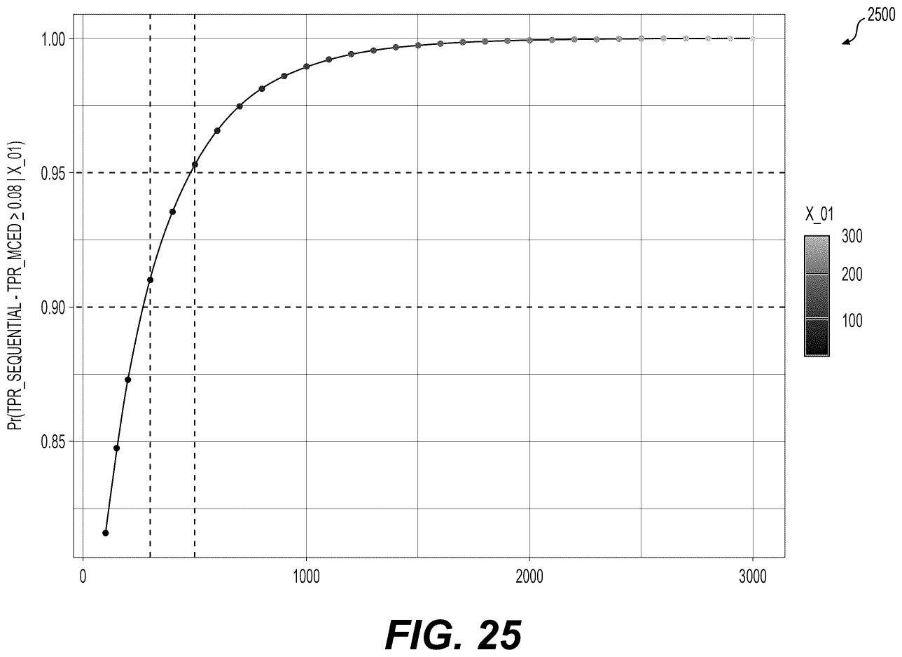

Referring now to FIG. 25, graph 2500 illustrates a paired study design simulation evaluating the TPR performance of a sequential testing system compared to a single MCED test, according to one or more aspects of the present disclosure. The x-axis represents the sample size, and the y-axis shows the posterior probability that the sequential test achieves at least an 8% improvement in TPR over the initial test, given an observed true difference of 10%. The scale represents the number of discordant positive results (X₀₁) observed between the two tests. The dashed horizontal lines indicate the 90% and 95% posterior probability thresholds, while the vertical dashed lines indicate sample sizes of approximately 300 and 500 individuals (the points where the poster probability exceeded the 90% and 95% thresholds, respectively). The curve of graph 2500 demonstrates that as sample size increases, the posterior probability of confirming a significant TPR improvement rapidly approaches 1.0, surpassing 95% confidence with fewer than 500 samples. These results indicate that the paired design may efficiently detect meaningful gains in sensitivity for sequential testing, providing strong statistical evidence even at moderate study scales.

Table 6 provided below demonstrates how introduction of sequential testing with multiple target specificities improves the performance of the overall test classification method. In this example, the low specificity target is fixed, while the high specificity target is increased to control the sensitivity and improve the PPV of the overall method. Correspondingly, the “High Spec (target)” column represents the target specificity of a given test, the “Low Spec (target)” column represents the specificity of the initial test combined with the high spec test (if applicable), the “High PPV” column and “Low PPV” column correspond to the estimated PPV of the high and low specificity tests, respectively. The “Overall Spec”, “Overall Sens”, “Overall PPV”, and “Overall Yield” columns show the values associated with the test including the combination of the low and high specificity test if applicable. As before, “Yield” metric, represents the proportion of true cancers identified in the screened population and is fixed, corresponding to the low target specificity initial test. However, overall specificity and overall PPV rise has the high target specificity rises. For this example, it is assumed that the retest negative rate for non-cancer is 80% and for cancers is 0%. These assumptions are based on empirical results and therefore represent realistic performance. As a reminder, this example is based on the testing schema discussed herein, so participants whose p (cancer) are above the threshold associated with the high specificity target in the first test are called positive, participants whose p (cancer) are between the threshold associated with the high specificity target and the low specificity target in the first test are retested, and participants whose p (cancer) are above the threshold associated with the low specificity target in the retest are called positive. The first two rows are associated with baseline values associated with a single point-in-time test.

TABLE 6

| High | Low | | | | | | |
|---|---|---|---|---|---|---|---|
| Spec | Spec | High | Low | Overall | Overall | Overall | Overall |
| (target) | (target) | PPV | PPV | Spec | Sens | PPV | Yield |
| 99.5 | N/A | N/A | N/A | 99.6 | 51.9 | 60.5 | 0.62 |
| 99.0 | N/A | N/A | N/A | 90.07 | 58.1 | 42.15 | 0.673 |
| 99.5 | 98.5 | 60.5 | 32.2 | 99.39 | 61.33 | 53.51 | 0.70 |
| 99.6 | 98.5 | 67.2 | 32.2 | 99.48 | 61.33 | 57.34 | 0.70 |
| 99.7 | 98.5 | 73.1 | 32.2 | 99.53 | 61.33 | 59.93 | 0.70 |
| 99.8 | 98.5 | 81.5 | 32.2 | 99.59 | 61.33 | 63.36 | 0.70 |

In general, any process discussed in this disclosure that is understood to be computer-implementable may be performed by one or more processors of a computer system, such as system environment 1510, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer server. A processor may be a central processing unit (CPU), a graphics processing unit (GPU), or any suitable type of processing unit.

A computer system, such as system environment 1510, may include one or more computing devices. If the one or more processors of the computer system are implemented as a plurality of processors, the plurality of processors may be included in a single computing device or distributed among a plurality of computing devices. If a system environment comprises a plurality of computing devices, the memory of the computer system may include the respective memory of each computing device of the plurality of computing devices.

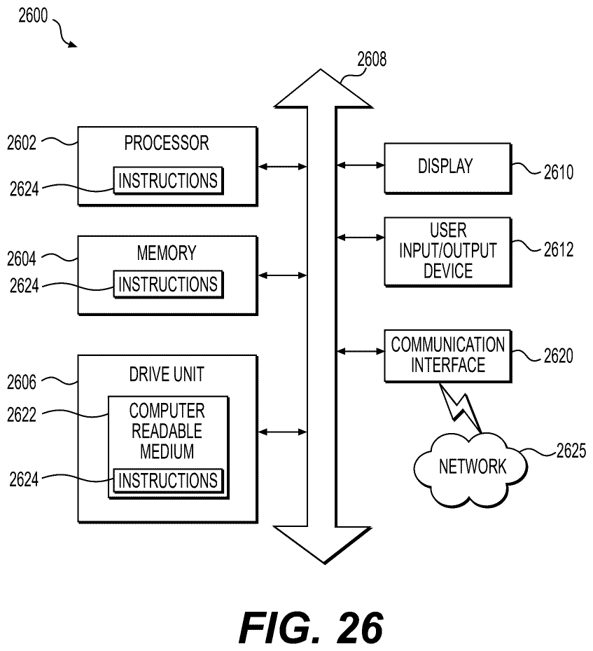

FIG. 26 is a simplified functional block diagram of a computer system 2600 that may be configured as a computing device for executing the processes described herein, according to exemplary embodiments of the present disclosure. In various embodiments, any of the systems herein may be an assembly of hardware including, for example, a data communication interface 2620 for packet data communication. The platform also may include a central processing unit (“CPU”) 2602, in the form of one or more processors, for executing program instructions. The platform may include an internal communication bus 2608, and a storage unit 2606 (such as ROM, HDD, SDD, etc.) that may store data on a computer readable medium 2622, although the system 2600 may receive programming and data via network communications via electronic network 2625 (e.g., voice, video, audio, images, or any other data over the electronic network 2625). The system 2600 may also have a memory 2604 (such as RAM) storing instructions 2624 for executing techniques presented herein, although the instructions 2624 may be stored temporarily or permanently within other modules of system 2600 (e.g., processor 2602 and/or computer readable medium 2622). The system 2600 also may include input and output ports 2612 and/or a display 2610 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

In this disclosure, the term “based on” means “based at least in part on.” The singular forms “a,” “an,” and “the” include plural referents unless the context dictates otherwise. The term “exemplary” is used in the sense of “example” rather than “ideal.” The terms “comprises,” “comprising,” “includes,” “including,” or other variations thereof, are intended to cover a non-exclusive inclusion such that a process, method, or product that comprises a list of elements does not necessarily include only those elements, but may include other elements not expressly listed or inherent to such a process, method, article, or apparatus. Relative terms, such as “about,” “approximately,” “substantially,” and “generally,” are used to indicate a possible variation of ±10% of a stated or understood value. In addition, the term “between” used in describing ranges of values is intended to include the minimum and maximum values described herein. The use of the term “or” in the claims and specification is used to mean “and/or” unless explicitly indicated to refer to alternatives only if the alternatives are mutually exclusive, although the disclosure supports a definition that refers to only alternatives and “and/or.” As used herein “another” may mean at least a second or more.

As used herein, the term “user” generally encompasses any person or entity, such as a researcher, laboratory technician, and/or a care provider (e.g., a doctor, etc.), that may desire information, resolution of an issue, or engage in any other type of interaction with a provider of the systems and methods described herein (e.g., via an application interface resident on their electronic device, etc.). The term “electronic application” or “application” may be used interchangeably with other terms like “program,” or the like, and generally encompasses software that is configured to interact with, modify, override, supplement, or operate in conjunction with other software.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for improving the accuracy of cancer screening in a population, the computer-implemented method comprising:
receiving, at a computer system, a first cancer test result associated with a first test performed on a first sample associated with a subject at a first time, wherein the first cancer test result is one of: a cancer signal detected (CSD) result, an indeterminate signal result, or a non-cancer signal detected (NCSD) result;
generating, responsive to receiving the indeterminate signal result, an instruction to perform a second test on the subject at a second time after a predetermined time interval;
receiving, at the computer system, a second cancer test result associated with the second test performed on a second sample associated with the subject at the second time, wherein the second cancer test result is one of: a persistent CSD result or the NCSD result; and
identifying, using a processor associated with the computer system, the subject as having cancer if the second cancer test result is the persistent CSD result; or
identifying, using the processor, the subject as not having cancer if the second cancer test result is the NCSD result.

2. The computer-implemented method of claim 1, wherein the predetermined time interval falls in a range of two week to six weeks.

3. The computer-implemented method of claim 1, wherein the first cancer test result and the second cancer test result are both generated using a multi-cancer early detection (MCED) assay.

4. The computer-implemented method of claim 3, wherein the MCED assay is based on assessment of cell-free DNA methylation.

5. The computer-implemented method of claim 1, wherein generating the instruction comprises only generating the instruction if the first cancer test result exceeds a lower decision boundary corresponding to an indeterminate range.

6. The computer-implemented method of claim 1, wherein identifying the subject as having cancer further comprises confirming that the cancer signal detected result exceeds a defined confidence threshold based on sequential test correlation.

7. The computer-implemented method of claim 1, wherein the identifying the subject as having cancer further comprises generating an automated clinical report comprising at least one of: a classifier score, a cancer signal origin, a tumor fraction estimate, and one or more persistent confidence values.

8. The computer-implemented method of claim 1, wherein identifying the subject as not having cancer further comprises determining that a cell-free DNA tumor fraction remains below a predefined limit of detection threshold.

9. The computer-implemented method of claim 1, further comprising classifying the second cancer test result as a true positive if a consistent tumor methylation signal is detected across a predefined number of CpG sites.

10. The computer-implemented method of claim 1, further comprising dynamically adjusting the predetermined time interval based on a historical cell-free DNA tumor fraction trend associated with the subject, cancer risk factors associated with the subject, or clinical information associated with the subject.

11. A system, comprising:
one or more processors;
one or more computer readable media storing instructions that are executable by the one or more processors to perform operations including:
receiving a first cancer test result associated with a first test performed on a first sample associated with a subject at a first time, wherein the first cancer test result is one of: a cancer signal detected (CSD) result, an indeterminate signal result, or a non-cancer signal detected (NCSD) result;
generating, responsive to receiving the indeterminate signal result, an instruction to perform a second test on the subject at a second time after a predetermined time interval;
receiving a second cancer test result associated with the second test performed on a second sample associated with the subject at the second time, wherein the second cancer test result is one of: a persistent CSD result or the NCSD result; and
identifying the subject as having cancer if the second cancer test result is the persistent CSD result; or
identifying the subject as not having cancer if the second cancer test result is the NCSD result.

12. The system of claim 11, wherein the predetermined time interval falls in a range of two week to six weeks.

13. The system of claim 11, wherein the first cancer test result and the second cancer test result are both generated using a multi-cancer early detection (MCED) assay.

14. The system of claim 13, wherein the MCED assay is based on assessment of cell-free DNA methylation.

15. The system of claim 11, wherein the instructions executable by the one or more processors for generating the instruction comprise instructions executable by the one or more processors for:
only generating the instruction if the first cancer test result exceeds a lower decision boundary corresponding to an indeterminate range.

16. The system of claim 11, wherein the instructions executable by the one or more processors for identifying the subject as having cancer further comprise instructions executable by the one or more processors for:
confirming that the cancer signal detected result exceeds a defined confidence threshold based on sequential test correlation.

17. The system of claim 11, wherein the instructions executable by the one or more processors for identifying the subject as having cancer further comprise instructions executable by the one or more processors for:
generating an automated clinical report comprising at least one of: a classifier score, a tumor fraction estimate, and one or more persistent confidence values.

18. The system of claim 11, wherein the instructions executable by the one or more processors for identifying the subject as having cancer further comprise instructions executable by the one or more processors for:
determining that a cell-free DNA tumor fraction remains below a predefined limit of detection threshold.

19. The system of claim 11, wherein the instructions are further executable by the one or more processors to perform operations including:
classifying the second cancer test result as a true positive if a consistent tumor methylation signal is detected across a predefined number of CpG sites.

20. A non-transitory computer-readable medium storing computer-executable instructions which, when executed by a system, cause the system to perform operations comprising:
receiving, at the system, a first cancer test result associated with a first test performed on a first sample associated with a subject at a first time, wherein the first cancer test result is one of: a cancer signal detected (CSD) result, an indeterminate signal result, or a non-cancer signal detected (NCSD) result;
generating, responsive to receiving the indeterminate signal result, an instruction to perform a second test on the subject at a second time after a predetermined time interval;
receiving, at the system, a second cancer test result associated with the second test performed on a second sample associated with the subject at the second time, wherein the second cancer test result is one of: a persistent CSD result or the NCSD result; and
identifying, using a processor associated with the system, the subject as having cancer if the second cancer test result is the persistent CSD result; or
identifying, using the processor, the subject as not having cancer if the second cancer test result is the NCSD result.

21. A computer-implemented method for enhancing a positive predictive value of cancer screening in a population, the computer-implemented method comprising:
receiving, at a computer system, a first cancer test result associated with a subject for a first cancer test, wherein the first cancer test was performed at a first time;
categorizing, using a processor associated with the computer system and based on the first cancer test result, the subject into at least one of a first group, a second group, and a third group, wherein the first group corresponds to a cancer signal value above a first threshold value, wherein the second group corresponds to a cancer signal value below a second threshold value, and wherein the third group corresponds to a cancer signal value between the first threshold value and the second threshold value;
generating, using the processor, a first recommendation to perform a diagnostic workup if the first cancer test result is associated with the first group;
generating, using the processor, a second recommendation to perform a second test on the subject if the first cancer test result is associated with the third group;
receiving, at the computer system, a second cancer test result associated with the subject for a second cancer test, wherein the second cancer test result is one of: a persistent cancer signal detected (CSD) result or a non-cancer signal detected (NCSD) result; and
identifying, using the processor, the subject as having cancer if the second cancer test result is the persistent CSD result; or
identifying, using the processor, the subject as not having cancer if the second cancer test result is the NCSD result.

22. The computer-implemented method of claim 21, wherein the first cancer test is configured to operate at a lower target specificity compared to the second cancer test.
